(19) United States
(12) Patent Application Publication
(10) Pub. No.: US 2013/0065807 A1
(43) Pub. Date: Mar. 14, 2013

(54) FRAGRANCE SUBSTANCE MIXTURES CONTAINING CERTAIN ISOLONGIFOLENYL METHYL ETHERS

(71) Applicants: Marcus Eh, Holzminden (DE); Pierre Kurzenne, Bois Colombes (FR); Marc vom Ende, Bodenwerder (DE); Emilie Coppermann, Paris (FR)

(72) Inventors: Marcus Eh, Holzminden (DE); Pierre Kurzenne, Bois Colombes (FR); Marc vom Ende, Bodenwerder (DE); Emilie Coppermann, Paris (FR)

(73) Assignee: SYMRISE AG, Holzminden (DE)

(21) Appl. No.: 13/607,187

(22) Filed: Sep. 7, 2012

(30) Foreign Application Priority Data
Sep. 9, 2011 (DE) 10 2011 082 464.2

(51) Int. Cl. C11D 3/50; A61K 8/35; A61Q 19/10; A61K 8/34; A61Q 13/00; A61Q 5/02; A61K 8/33; A61K 8/49

(52) U.S. Cl. 510/104; 512/19; 512/17; 512/11; 512/12

(57) ABSTRACT

The present invention primarily relates to fragrance substance mixtures, preferably perfume oils, comprising
(a) compound (A-1) and compound (A-2)

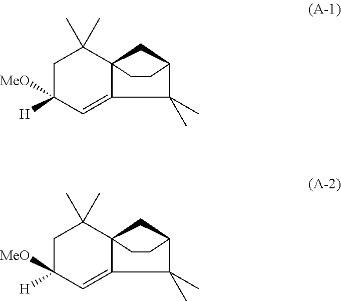 [SMILES: CO[C@H]1C=C2C(C)(C)[C@H]3CC[C@@]2(C3)C(C)(C)C1 ; CO[C@@H]1C=C2C(C)(C)[C@H]3CC[C@@]2(C3)C(C)(C)C1]

and
(b) one or more further musk fragrance substances, which are not isolongifolenyl methyl ethers,
   wherein the mass ratio of the total quantity of musk fragrance substances of constituent (b) to the total quantity of compounds (A-1) and (A-2) (constituent (a)) is preferably greater than or equal to 1:2.

Furthermore, the present invention relates to perfumed products containing fragrance substance mixtures of this type according to the invention, in particular perfume oils, and a method for modifying certain olfactory properties of fragrance substances, fragrance substance mixtures, perfume oils or correspondingly perfumed products.

The present invention primarily relates to fragrance substance mixtures, preferably perfume oils, comprising
(a) compound (A-1) and compound (A-2)

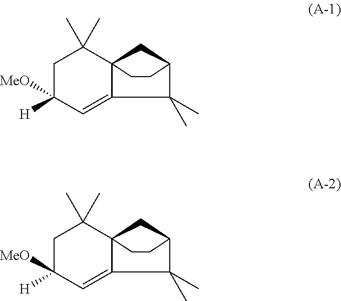 [SMILES: CO[C@H]1C=C2C(C)(C)[C@H]3CC[C@@]2(C3)C(C)(C)C1 ; CO[C@@H]1C=C2C(C)(C)[C@H]3CC[C@@]2(C3)C(C)(C)C1]

and
(b) one or more further musk fragrance substances, which are not isolongifolenyl methyl ethers,
   wherein the mass ratio of the total quantity of musk fragrance substances of constituent (b) to the total quantity of compounds (A-1) and (A-2) (constituent (a)) is preferably greater than or equal to 1:2.

Furthermore, the present invention relates to perfumed products containing fragrance substance mixtures of this type according to the invention, in particular perfume oils, and a method for modifying certain olfactory properties of fragrance substances, fragrance substance mixtures, perfume oils or correspondingly perfumed products.

Diverse isolongifolenyl alkyl ethers and their olfactory properties are described in EP 1 178 105. EP 1 178 105 also discloses isolongifolenyl methyl ethers, also including the two isomeric compounds (A-1) and (A-2). Ambergris, dry musk, slightly spicy and weakly woody patchouli are given there as the odour evaluation for the stereoisomer (A-1). Stereoisomer (A-2) is characterised there as a fragrance substance with an odour of musk, slightly floral, cashmeran, exalting and spicy. EP 1 178 105 furthermore discloses an odour of strongly musk, powdery, cashmeran, ambergris, exalting and slightly woody for a mixture comprising 37.6 GC-% (A-1) and 59.1 GC-% (A-2).

Mixtures or combinations of these isolongifolenyl alkyl ethers with further musk fragrance substances, which are not isolongifolenyl alkyl ethers, are not disclosed in EP 1 178 105. EP 1 178 105 discloses, in particular, no quantity ratios and no olfactory effects in the combination of these isolongifolenyl alkyl ethers with musk fragrance substances that are structurally different from them.

There is repeatedly a need in perfumery to modify, to emphasise (i.e. intensify or enhance) or to mask (i.e. to reduce or suppress) certain olfactory aspects of a fragrance substance or a fragrance substance mixture.

Perfume oils and perfumed products containing one or more musk fragrance substances are known to the person skilled in the art in the perfumery area, see, for example, EP 1 552 814.

Musk fragrance substances are known to the person skilled in the art and play a very important part in perfumery. The person skilled in the art in the perfumery area knows that in musk fragrance substances, in particular macrocyclic musk fragrance substances, apart from their typical desired main notes, which bring about an erogenous, masculine and animalistic odour, undesired secondary notes can occur. Such undesired and disturbing secondary notes, also called faulty notes or off notes, are metallic notes, in particular notes of hot pressing irons (called pressing iron note below), such as also described for musk fragrance substances in EP 1 487 816 and EP 1 220 850.

A further disadvantageous effect, which can be observed in conjunction with musk fragrance substances in a fragrance substance composition or a perfume oil, is the lowering or reduction of the odour intensity (i.e. the total odour intensity) of a fragrance substance composition of this type or a perfume oil of this type. There is therefore also regularly the necessity to counteract the lowering or reduction of the odour intensity of a fragrance substance composition of this type or a perfume oil of this type by adding odourintensifying substances (so-called “enhancers” or “boosters”).

The primary object of the present invention was to find a substance (substance or substance mixture), capable of modifying, preferably masking, i.e. reducing or suppressing, the undesired olfactory notes of musk fragrance substances, in particular of macrocyclic musk fragrance substances. These undesired notes are, in particular, metallic notes, in particular odour notes of a hot pressing iron.

In addition to the modifying or masking effect, the substance looked for should preferably also be able to counteract the lowering or reduction of the odour intensity (i.e. the total odour intensity) of a fragrance substance composition of this type or a perfume oil of this type regularly observed in conjunction with musk fragrance substances in a fragrance substance composition or a perfume oil. It should therefore preferably be possible in this regard to bring about an olfactory intensification or emphasis (“enhancer” or “booster” effect).

This object is achieved by a fragrance substance mixture according to the invention, preferably by a perfume oil according to the invention, comprising
(a) compound (A-1) and compound (A-2)

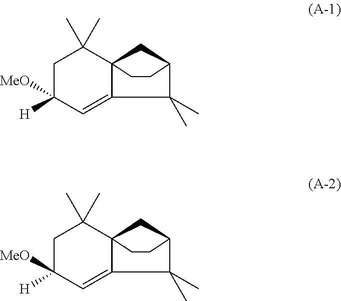 [SMILES: CO[C@H]1C=C2C(C)(C)[C@H]3CC[C@@]2(C3)C(C)(C)C1 ; CO[C@@H]1C=C2C(C)(C)[C@H]3CC[C@@]2(C3)C(C)(C)C1]

and
(b) one or more further musk fragrance substances, which are not isolongifolenyl methyl ethers, preferably one or more further musk fragrance substances, which are not isolongifolenyl-C₁-C₄-alkyl ethers
   wherein the mass ratio of the total quantity of musk fragrance substances of constituent (b) to the total quantity of compounds (A-1) and (A-2) (constituent (a)) is preferably greater than or equal to 1:2, preferably greater than or equal to 1:1, more preferably greater than or equal to 3:2.

### Constituent (a) of a Fragrance Substance Mixture According to the Invention:

Constituent (a) of a fragrance substance mixture according to the invention is a diastereomeric mixture. Constituent (a) consists of the two epimeric compounds (A-1) and (A-2).

The compounds (A-1) and (A-2) of the structure formulas given above are certain isomers of the isolongifolenyl methyl ether and known per se from the literature. They may, for example, be produced as described in EP 1 178 105 (see above).

The mass ratio of the total quantity of the isomer (A-1) to the total quantity of the isomer (A-2) is preferably greater than or equal to 11:4, preferably greater than or equal to 3:1, more preferably greater than or equal to 7:2.

The mass ratio of the total quantity of the isomer (A-1) to the total quantity of the isomer (A-2) is preferably in the range from 3:1 to 12:1, more preferably in the range from 7:2 to 10:1, particularly preferably in the range from 15:4 to 10:1, and most preferably in the range from 4:1 to 8:1.

The isomeric mixtures to be used according to the invention of the compounds (A1) and (A2), were produced within the framework of the present invention analogously to the methods described in EP 1 178 105. Such isomeric mixtures were produced by means of fractionated distillation of the corresponding reaction product mixture, wherein, depending on the distillation conditions, a plurality of different fractions of isomeric mixtures to be used according to the invention were obtained, which can be used according to the invention as constituent (a). Alternatively, the compounds (A1) and (A2) were firstly produced in (substantially) pure form and then the desired weight ratio of (A1) to (A2) was adjusted by mixing the corresponding quantity fractions of the two pure compounds (A1) and (A2).

The preferred embodiments of fragrance substance mixtures according to the invention, in particular the mass ratios designated preferred or particularly preferred, apply likewise and correspondingly to perfume oils according to the invention, perfumed products according to the invention and to methods according to the invention.

The modifying effects described in conjunction with the present invention, in particular the masking and intensifying effects, are observed most strongly and clearly when using the compounds (A-1) and (A-2) in the quantity ratios in relation to one another designated preferred or particularly preferred, i.e. the preferred isomeric mixtures given of the compounds (A-1) and (A-2) of the constituent (a).

These described effects also occur particularly significantly when the isomeric mixtures mentioned of the compounds (A-1) and (A-2) are combined with further constituents of a fragrance substance mixture according to the invention, a perfume oil according to the invention or a product perfumed according to the invention, in particular in the embodiments designated preferred or particularly preferred below.

### Constituent (b) of a Fragrance Substance Mixture According to the Invention:

The, or the further, musk fragrance substance(s) of the constituent (b) are fragrance substances which have a musk odour. Fragrance substances of this type are known to the person skilled in the art, as musk is a very important odour orientation in perfumery.

The further musk fragrance substances of constituent (b) are not isolongifolenyl methyl ethers and preferably not isolongifolenyl-C₁-C₄-alkyl ethers.

Isolongifolenyl-C₁-C₄-alkyl ethers (in accordance with the structure formula shown on the left below) or isolongifolenyl methyl ethers (in accordance with the structure formula shown on the right below) are taken to mean, in the scope of the present invention, all compounds, including all stereoisomers, of the following structure formulas:

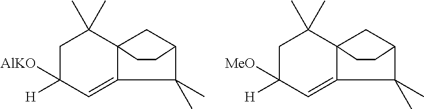

wherein the radical “Alk” stands for an alkyl radical with 1 to 4 carbon atoms and “Me” (as generally conventional) signifies a methyl group.

The further musk fragrance substances of constituent (b) therefore differ from constituent (a).

According to the invention, the mass ratio of the total quantity of further musk fragrance substances of constituent (b) to the total quantity of compounds of constituent (a) is in the range from 3:2 to 20:1, preferably in the range from 3:2 to 15:1, particularly preferably in the range from 2:1 to 12:1.

Fragrance substance mixtures preferred according to the invention, preferably perfume oils, contain two, three, four, five or more different musk fragrance substances as constituent (b), each of the musk fragrance substances preferably being selected independently of the other musk fragrance substances from the group of macrocyclic musk fragrance substances, the nitro-musk fragrance substances (aromatic musk fragrance substances with nitro group(s)), polycyclic musk fragrance substances and/or alicyclic musk fragrance substances.

The or the further musk fragrance substance(s) of constituent (b) are preferably selected from the group of macrocyclic musk fragrance substances, the nitro-musk fragrance substances (aromatic musk fragrance substances with nitro group(s)), polycyclic musk fragrance substances and/or alicyclic musk fragrance substances, preferably one, two, three or more musk fragrance substances from the following Table 1.

Fragrance substance mixtures according to the invention containing the compounds (A-1) and (A-2) and one or more further musk fragrance substances of constituent (b), preferably in which the mass ratio of the total quantity of further musk fragrance substances of constituent (b) to the total quantity of compounds (A-1) and (A-2) (constituent (a)) is in the quantity ratios given above (in particular given as preferred or particularly preferred), exhibit a modified, overall improved odour quality with regard to the undesired notes of the musk fragrance substances of constituent (b).

In comparison to an otherwise identically composed comparative fragrance substance mixture, which does not contain the compounds (A-1) and (A-2), the fragrance substance mixtures according to the invention exhibit less pronounced metallic notes, i.e. the metallic notes, in particular the pressing iron note, of the musk fragrance substances of constituent (b) are masked, i.e. partially or even completely reduced, by the presence of compounds (A-1) and (A-2) of the constituent (a).

In addition, by adding the compounds (A-1) and (A-2) a certain, mostly moderate to averagely strongly pronounced increase in the odour intensity (i.e. the total odour intensity) of a fragrance substance mixture containing one or more musk fragrance substances can be achieved. The epimeric compounds (A-1) and (A-2) act, in particular in the isomeric ratios given above, as “enhancers” or “boosters” for musk fragrance substances and for fragrance substance mixtures and perfume oils containing one or more musk fragrance substances.

The musk fragrance substance(s) in the scope of the present invention are preferably selected from the following Table 1:

TABLE 1

| | Product/Product name/ | |
|---|---|---|
| TYPE | Brand name | Name(s)/CAS name |
| MACRO | EXALTENONE | 4-Cyclopentadecen-1-one (4Z)-; 4- |
| | | Cyclopentadecen-1-one |
| MACRO | CYCLOHEPTADECENONE | 9-Cycloheptadecen-1-one |
| MACRO | CIVETONE | 9-Cycloheptadecen-1-one, (9Z)- |
| MACRO | CYCLOHEXADECANOLIDE, | Oxacycloheptadecan-2-one, |
| | DIHYDROAMBRETTOLIDE | ω-Hexadecanolide |
| MACRO | ETHYLENE | 1,4-Dioxacyclohexadecane-5,16-dione |
| | DODECANDIOATE | |
| MACRO | GLOBALIDE ® | Oxacyclohexadecen-2-one; 15-Pentadec- |
| | | (11/12)-enolide |
| MACRO | ETHYLENE BRASSYLATE | 1,4-Dioxacycloheptadecane-5,17-dione |
| MACRO | MUSCONE | 3-Methy-cyclopentadecanone |
| MACRO | AMBRETTOLIDE | Oxacycloheptadec-10-en-2-one |
| MACRO | MUSCENONE | 3-Methyl-cyclopentadecenone |
| MACRO | VELVIONE ®, AMBRETONE | 5-Cyclohexadecen-1-one |
| MACRO | AURELIONE ® | 7/8-Cyclohexadecen-1-one |
| MACRO | GLOBANONE ® | 8-Cyclohexadecen-1-one |
| MACRO | ISOMUSCONE ® | Cyclohexadecanone |
| MACRO | EXALTOLIDE ®, | 15-Pentadecanolide, Oxacyclohexadecan- |
| | MACROLIDE ® | 2-one, 16-Oxacyclohexadecan-1-one |
| MACRO | COSMONE ® | 3-Methyl-(5E/Z)-cyclotetradecen-1-one |
| POLY | TRASEOLIDE ® | 1[2,3-Dihydro-1,1,2,6-tetramethyl-3-(1- |
| | | methylethyl)-1H-inden-5-yl]-ethanone |
| POLY | PHANTOLIDE ® | 1-(2,3-Dihydro-1,1,2,3,3,6-hexamethyl-1H- |
| | | inden-5-yl)ethanone |
| POLY | TONALIDE ® | 1-(5,6,7,8-Tetrahydro-3,5,5,6,8,8- |
| | | hexamethyl-2-naphthalenyl)-ethanone |
| POLY | CRYSOLIDE | 1-[6-(1,1-Dimethylethyl)-2,3-dihydro-1,1- |
| | | dimethyl-1H-inden-4-yl]-ethanone |
| POLY | CHROMANOLIDE ® | Tetradecanoic acid, 1-methylethyl ester; |
| | | Cyclopenta[g]-2-benzopyrane, 1,3,4,6,7,8- |
| | | hexahydro-4,6,6,7,8,8-hexamethyl- |
| POLY | GALAXOLIDE ® | Cyclopenta[g]-2-benzopyrane, 1,3,4,6,7,8- |
| | | hexahydro-4,6,6,7,8,8-hexamethyl- |
| ALICYC | HELVETOLIDE ® | 1-Propanol, 2-[1-(3,3- |
| | | dimethylcyclohexyl)ethoxy]-2-methyl-, 1- |
| | | propanoate |
| NITRO | MOSKENE | 2,3-Dihydro-1,1,3,3,5-pentamethyl-4,6- |
| | | dinitro-1H-indene |
| NITRO | MUSK TIBETENE | 1-(1,1-Dimethylethyl)-3,4,5-trimethyl-2,6- |
| | | dinitrobenzene |
| NITRO | ORINOX | 1-[4-(1,1-Dimethylethyl)-2,6- |
| | | dimethylphenyl]-ethanone |
| NITRO | MUSK XYLOL | 1-(1,1-Dimethylethyl)-3,5-dimethyl-2,4,6- |
| | | trinitrobenzene |
| NITRO | MUSK KETONE | 1-[4-(1,1-Dimethylethyl)-2,6-dimethyl-3,5- |
| | | dinitrophenyl]-ethanone |
| NITRO | MUSK ALPHA | 1,3-Dibromo-2-methoxy-4-methyl-5- |
| | | nitrobenzene |

MACRO = macrocyclic musk fragrance substances
NITRO = Nitro musk fragrance substances
POLY = polycyclic musk fragrance substances
ALICYC = alicyclic musk fragrance substance

Further preferred as part of constituent (b) are polycyclic and/or macrocyclic musk fragrance substances, whereupon macrocyclic musk fragrance substances, in particular, have proven to be particularly advantageous in the sense of the invention, which are preferably in turn selected from the group consisting of macrocyclic C₁₄-C₁₈ ketones and macrocyclic C₁₄-C₁₈ lactones, the ketone or lactone having a ring size of 15 to 17 ring atoms and having no, one or two oxygen atoms in the ring.

Most preferred are fragrance substance mixtures according to the invention, the constituent (b) of which comprises or consists of:
(b) 15-pentadec-(11/12)-enolide, ethylene brassylate, 15-pentadecanolide, 5-cyclohexadecen-1-one, 7-cyclohexadecen-1-one, 8-cyclohexadecen-1-one, 7/8-cyclohexadecen-1-one, cyclohexadecanone, 3-methylcyclopentadecanone, 3-methylcyclopentadecanone and/or 9-cycloheptadecen-1-one and mixtures thereof.

In a preferred embodiment, constituent (b) is selected from the group consisting of 15-pentadec-(11/12)-enolide (Globalide®), ethylene brassylate and oxacyclohexadecan-2-one (Macrolide®) and mixtures thereof.

According to the above statements, the present invention relates to a method for modifying, preferably for reducing or suppressing, a metallic note of one or more musk compounds, preferably of one or more macrocyclic musk compounds, comprising the following step:
mixing one or more musk compounds, preferably one or more macrocyclic musk compounds, with a total quantity of compounds (A-1) and (A-2) (constituent (a)), as defined above, which is sufficient to modify, preferably to reduce or suppress, the metallic note, in particular the pressing iron note, of one, several or all the musk compounds.

### Optional Constituent (c) of a Fragrance Substance Mixture According to the Invention:

A fragrance substance mixture according to the invention, preferably a perfume oil according to the invention, in addition to the constituents (a) and (b), preferably contains as a further constituent
(c) 1, 2, 3, 4, 5 or more terpene alcohols having the summation formula C₁₀HₓO, wherein x signifies the number 18, 20 or 22 and wherein preferably one, several or all the terpene alcohols are selected from the group consisting of linalool, eucalyptol, dihydromyrcenol, citronellol, nerol, geraniol, menthol, tetrahydro linalool and tetrahydro geraniol.

A fragrance substance mixture according to the invention, preferably a perfume oil according to the invention, in addition to the constituents (a) and (b), preferably contains, as a further constituent
(c) 1, 2, 3, 4, 5 or more terpene alcohols selected from the group consisting of linalool, eucalyptol, dihydromyrcenol, citronellol, nerol, geraniol, menthol, tetrahydro linalool and tetrahydro geraniol, preferably 1, 2, 3, 4 or all the terpene alcohols selected from the group consisting of linalool, dihydromyrcenol, citronellol, nerol and geraniol.

In a fragrance substance mixture according to the invention, preferably a perfume oil according to the invention, the mass ratio of the total quantity of terpene alcohols (constituent (c)) to the total quantity of compounds (A-1) and (A-2) (constituent (a)) is greater than or equal to 1:1, preferably greater than or equal to 2:1, more preferably greater than or equal to 3:1, particularly preferably greater than or equal to 4:1.

The mass ratio of the total quantity of terpene alcohols (constituent (c)) to the total quantity of compounds (A-1) and (A-2) (constituent (a)) is preferably in the range from 3:1 to 15:1, particularly preferably in the range from 4:1 to 10:1.

In particular when adjusting the aforementioned mass ratios of the total quantity of terpene alcohols (constituent (c)) to the total quantity of compounds (A-1) and (A-2) (constituent (a)) in a fragrance substance mixture according to the invention or a perfume oil according to the invention, the olfactory quality is improved overall. By adding constituent (c) not only are the odour notes particular to the terpene alcohol(s) of constituent (c) added to a fragrance substance mixture according to the invention or a perfume oil according to the invention, but an odour profile going beyond this and more complex overall is produced. Furthermore, an unexpected olfactory rounding and a further intensification of the odour impression can be observed.

The terpene alcohols of constituent (c) may be of natural or synthetic origin. In the case of a natural origin, they may be part of a corresponding essential oil or have been enriched or isolated from one such.

### Optional Constituent (d) of a Fragrance Substance Mixture According to the Invention:

A fragrance substance mixture according to the invention, preferably a perfume oil according to the invention, in addition to constituents (a) and (b) and optionally (c), preferably contains as a further constituent
(d) 1, 2, 3 or more further woody fragrance substances (wood fragrance substances), wherein the further woody fragrance substance or the further woody fragrance substances of constituent (d) is or are different from constituent (a) and constituent (b).

The wood fragrance substances of constituent (d) of a fragrance substance mixture according to the invention or a perfume oil according to the invention, in combination with the constituents (a) and (b) (and optionally constituent (c)) of a fragrance substance mixture according to the invention or a perfume oil according to the invention, bring about a more harmonious, more natural, more lively (more animated) and overall more valuable odour impression.

Examples of woody fragrance substances, which may be part of the optional constituent (d) are known to the person skilled in the art and are to be found, for example, in S. Arctender, Perfume and Flavor Materials, Vol. I and II, Montclair, N.J., 1969, self-published or H. Surburg, J. Panten “Common Fragrance and Flavor Materials”, 5^{th} ed., Wiley-VCH, Weinheim 2006.

A fragrance substance mixture according to the invention, preferably a perfume oil according to the invention, preferably contains a sensorily (olfactorily) effective quantity of constituent (d), preferably in a quantity which is sufficient to give a fragrance substance mixture according to the invention, preferably a perfume oil according to the invention, a woody note, preferably a sandalwood note.

Preferred fragrance substance mixtures according to the invention, preferably perfume oils according to the invention, are those, in which constituent (d) comprises one, two, three or more further woody fragrance substances or consists thereof, selected from the group consisting of
(d) (i) 2,3,8,8-Tetramethyl-1,2,3,4,5,6,7,8-octahydro-2-naphthalenyl methyl ketone and/or 1-[1,2,3,4,6,7,8,8a-octahydro-1,2,8,8-tetramethylnaphtalen-2-yl]ethanone
   and/or
(d)(ii) sandalwood fragrance substances, preferably selected from the group consisting of 2-methyl-4-(2,2,3-trimethyl-3-cyclopenten-1-yl)butanol (for example Brahmanol), 5-(2,2,3-trimethyl-3-cyclopenten-1-yl)-3-methylpentan-2-ol (for example Sandalore®), 2-ethyl-4-(2,2,3-trimethyl-3-cyclopenten-1-yl)-2-buten-1-ol (for example Sandranol®), 3-methyl-5-(2,2,3-trimethyl-3-cyclopenten-1-yl)-4-penten-2-ol (for example Ebanol®), 3,3-dimethyl-5-(2,2,3-trimethyl-3-cyclopenten-1-yl)-4-penten-2-ol (for example Polysantol®), 3-isocamphylcyclohexanol (for example Sandel 80®) and [1-methyl-2-(1,2,2-trimethyl[3.1.0]hex-3-ylmethyl)cyclopropyl]methanol (for example Javanol®).

Fragrance substance mixtures according to the invention, in particular perfume oils according to the invention, comprising compounds (A-1) and (A-2) (constituent (a)), one or more further musk fragrance substances (constituent (b)) and one, two, three or more further woody fragrance substances (constituent (d)) exhibit a harmonisation and rounding of the odour picture.

Mixtures containing both compounds of constituent (d)(i) are, for example, known by the name Iso E Super® and commercially available.

In fragrance substance mixtures according to the invention, in particular perfume oils according to the invention, comprising compounds (A-1) and (A-2) (constituent (a)), one or more further musk fragrance substances (constituent (b)) and constituent (d)(i) 2,3,8,8-Tetramethyl-1,2,3,4,5,6,7,8-octahydro-2-naphthalenyl methyl ketone and/or 1-[1,2,3,4,6,7,8,8a-octahydro-1,2,8,8-tetramethylnaphtalen-2-yl]ethanone, in particular in the mass ratios given below, a more harmonious and more complex olfactory effect can be observed, which allows the fragrance substance mixture or the perfume oil to appear more valuable overall.

A further preferred embodiment relates to a fragrance substance mixture according to the invention, preferably a perfume oil according to the invention, wherein the mass ratio of the total quantity of constituent (d)(i) 2,3,8,8-Tetramethyl-1,2,3,4,5,6,7,8-octahydro-2-naphthalenyl methyl ketone and 1-[1,2,3,4,6,7,8,8a-octahydro-1,2,8,8-tetramethylnaphtalen-2-yl]ethanone to the total quantity of compounds (A-1) and (A-2) (constituent (a)) is greater than or equal to 1:1, preferably greater than or equal to 2:1, more preferably greater than or equal to 3:1, particularly preferably greater than or equal to 4:1.

In a preferred embodiment, the mass ratio of the total quantity of constituent (d)(i) to the total quantity of compounds (A-1) and (A-2) (constituent (a)) is in the range from 4:1 to 25:1, more preferably in the range from 5:1 to 20:1, particularly preferably in the range from 6:1 to 12:1.

A further preferred embodiment relates to a fragrance substance mixture according to the invention, preferably a perfume oil according to the invention, wherein the mass ratio of the total quantity of sandalwood fragrance substances (constituent (d)(ii)) to the total quantity of compounds (A-1) and (A-2) (constituent (a)) is greater than or equal to 1:1, preferably greater than or equal to 2:1, preferably greater than or equal to 3:1, more preferably greater than or equal to 4:1.

In a preferred embodiment, the mass ratio of the total quantity of constituent (d)(ii) to the total quantity of compounds (A-1) and (A-2) (constituent (a)) is in the range from 2:1 to 20:1, more preferably in the range from 3:1 to 15:1, particularly preferably in the range from 4:1 to 10:1.

Fragrance substance mixtures according to the invention, in particular perfume oils according to the invention, comprising compounds (A-1) and (A-2) (constituent (a)), one or more further musk fragrance substances (constituent (b)) and constituent (d), in particular constituent (d)(ii), exhibit, apart from an olfactory harmonisation and rounding, additionally creamier, softer and/or milkier notes and therefore overall a more elegant and (still) more valuable odour impression. In this case, in particular the enhancing of the woody softness and the creamy and milky notes of the sandalwood fragrance substances are noteworthy.

This applies, in particular, to the woody fragrance substances of constituent (d) preferably defined below and in particular to the woody substances explicitly mentioned as preferred, and in particular, the sandalwood fragrance substances of constituent (d)(ii) from the group consisting of 2-methyl-4-(2,2,3-trimethyl-3-cyclopenten-1-yl)butanol, 5-(2,2,3-trimethyl-3-cyclopenten-1-yl)-3-methylpentan-2-ol, 2-ethyl-4-(2,2,3-trimethyl-3-cyclopenten-1-yl)-2-buten-1-ol, 3-methyl-5-(2,2,3-trimethyl-3-cyclopenten-1-yl)-4-penten-2-ol, 3,3-dimethyl-5-(2,2,3-trimethyl-3-cyclopenten-1-yl)-4-penten-2-ol, 3-isocamphylcyclohexanol and [1-methyl-2-(1,2,2-trimethyl[3.1.0]hex-3-ylmethyl)cyclopropyl]methanol.

Preferred fragrance substance mixtures according to the invention, preferably perfume oils according to the invention, are those, in which constituent (c) contains one, two, three or more fragrance substances from the group consisting of (conventional trademark or product names are optionally disclosed in brackets):

Preferred further wood fragrance substances of constituent (d) of a fragrance substance mixture according to the invention, which are not to be assigned to the constituents (d)(i) and (d)(ii), are the following:

cedryl methyl ether (Cedramber), cedryl methyl ketone, cedryl acetate, (4aR,5R,7aS,9R)octahydro-2,2,5,8,8,9a-hexamethyl-4H-4-a,9-methanoazuleno(5,6-d)-1,3-dioxol) (Ambrocenide®), 1′,1′,5′,5′-Tetramethylhexahydro-spiro[1.3-dioxolan-2.8°(5′H)-2H-2.4a]methanonaphthalene (Ysamber® K), 1-(2,2,6-Trimethylcyclohexyl)hexan-3-ol (Timberol®), Cyclododecyl methyl ether (Palisandin), (Ethoxymethoxy)cyclododecane (Boisambrene Forte®), 3a,6,6,9a-Tetramethyldodecahydronaphtho[2,1-b]furan (Ambroxid®) and 2,2,7,7-Tetramethyltricyclo[6.2.1.0^{1,6}]undecan-6-ol (Terranol®).

In a preferred embodiment, a fragrance substance mixture according to the invention, preferably a perfume oil according to the invention, apart from the constituents (a) and (b), contains the constituent (c) and the constituent (d).

If a fragrance substance mixture according to the invention, a perfume oil according to the invention or a perfumed product according to the invention, contains a constituent (d), which simultaneously also fulfils the criteria of constituent (b), the latter, in particular for quantitative observations, is regarded as part of the constituent (b) and also assigned thereto.

A fragrance substance mixture according to the invention, a perfume oil according to the invention or a product according to the invention, is preferred, preferably in one of the embodiments given as preferred above, to which there applies:
the mass ratio of the total quantity of further musk fragrance substances of constituent (b) to the total quantity of compounds (A-1) and (A-2) (constituent (a)) is in the range from 3:2 to 20:1, preferably in the range from 3:2 to 15:1, particularly preferably in the range from 2:1 to 12:1,
and/or
the mass ratio of the total quantity of linalool, eucalyptol, dihydromyrcenol, citronellol, nerol, geraniol, menthol, tetrahydro linalool and tetrahydro geraniol ((preferred terpene alcohols of the constituent (c)) to the total quantity of compounds (A-1) and (A-2) (constituent (a)) is greater than or equal to 2:1, preferably greater than or equal to 3:1, and is more preferably in the range from 3:1 to 15:1, particularly preferably in the range from 4:1 to 10:1,
and/or
the mass ratio of the total quantity of constituent (d)(i) 2,3,8,8-Tetramethyl-1,2,3,4,5,6,7,8-octahydro-2-naphthalenyl methyl ketone and 1-[1,2,3,4,6,7,8,8a-octahydro-1,2,8,8-tetramethylnaphtalen-2-yl]ethanone to the total quantity of compounds (A-1) and (A-2) (constituent (a)) is greater than or equal to 2:1, preferably greater than or equal to 3:1, and is more preferably in the range from 4:1 to 25:1, more preferably in the range from 5:1 to 20:1, particularly preferably in the range from 6:1 to 12:1,
and/or
the total quantity of constituent (d)(ii) to the total quantity of compounds (A-1) and (A-2) (constituent (a)) is greater than or equal to 2:1 and is preferably in the range from 2:1 to 20:1, more preferably in the range from 3:1 to 15:1, particularly preferably in the range from 4:1 to 10:1,
   in each case based on the total fragrance substance mixture, the total perfume oil or the total perfumed product.

In a particularly preferred embodiment, two, three or all the above-mentioned preferred embodiments and/or mass ratios with respect to constituents (b), (c), (d)(i) and (d)(ii) are adjusted simultaneously, in turn in particular at least the (preferred or particularly preferred) mass ratios relating to constituent (b) being adjusted.

It may therefore be noted, to summarise, that compounds (A-1) and (A-2) (constituent (a)) together with the musk fragrance substance(s) of the constituent (b) advantageously, in combination with other fragrance substances, bring about certain olfactory aspects.

It can further be noted in view of that stated above that compounds (A-1) and (A-2) (constituent (a)) together with the musk fragrance substance(s) of constituent (b) advantageously, in combination with other fragrance substances, emphasise (enhance) certain olfactory aspects of these fragrance substances.

In accordance with the above statements, the present invention, in a further aspect, relates to a method for increasing the odour intensity of a fragrance substance mixture comprising one or more musk compounds, preferably comprising one or more macrocyclic musk compounds, having the following step:
mixing one or more musk components, preferably one or more macrocyclic musk compounds, with a total quantity of compounds (A-1) and (A-2) as defined above, which is sufficient to increase the odour intensity of the fragrance substance mixture.

In view of that stated above, it can also be noted that compounds (A-1) and (A-2) (constituent (a)), in particular together with the musk fragrance substance(s) of constituent (b), advantageously, in combination with other fragrance substances, emphasise (enhance) certain olfactory aspects of these fragrance substances.

In accordance with the statements above, the present invention, in a further aspect, relates to a method for emphasising (enhancing) or intensifying an odour with a creamy, soft and/or milky note, preferably of one or more sandalwood fragrance substances, having the following step:

mixing of
one or more fragrance substances, preferably selected from the compounds of constituent (d)(ii), preferably mentioned above, with a creamy, soft and/or milky note, in particular with a sandalwood note,
one or more musk fragrance substances of constituent (b), preferably selected from the musk fragrance substances of constituent (b) designated as preferred above,
and
a total quantity of compounds (A-1) and (A-2) (constituent (a)), which is sufficient to emphasise the odour impression of the fragrance substance(s), which cause a creamy, soft and/or milky note, in particular a sandalwood note.

The above details on preferred fragrance substance mixtures according to the invention, perfume oils and perfumed products apply accordingly to the methods according to the invention.

Fragrance substance compositions according to the invention (fragrance substance mixtures), preferably perfume oils according to the invention, may be used in liquid form, undiluted or diluted with a solvent for perfuming or aromatisation. Suitable solvents for this are, in particular, ethanol, glycerol, 1,2-propylene glycol, 1,2-butylene glycol, dipropylene glycol, diethyl phthalate, triethyl citrate, isopropyl myristate and triacetin.

Substances, with which fragrance substance mixtures according to the invention, in particular perfume oils, can be combined, are:

preservatives, preferably the abrasives, anti-acne agents and agents for sebum reduction mentioned in US 2006/0089413, preferably the agents mentioned in WO 2008/046791 against skin ageing, preferably the antibacterial agents, anti-cellulite agents, anti-dandruff agents mentioned in WO 2005/123101, preferably the anti-inflammatory agents, irritationpreventing agents, anti-irritants (anti-inflammatory, irritation-inhibiting and irritationpreventing agents) mentioned in WO 2008/046795, preferably the antimicrobial agents mentioned in WO 2007/042472 and US 2006/0089413, preferably the antioxidants mentioned in WO 2005/123101, preferably the astringents, antiseptic agents, antistatics, binders, buffers, carrier materials mentioned in WO 2005/123101, preferably the chelating agents mentioned in WO 2005/123101, preferably the cell stimulants, cleaning agents, care agents, depilatory agents, surfactant substances, deodorising agents and antiperspirants mentioned in WO 2005/123101, preferably the softeners, emulsifiers mentioned in WO 2005/123101, preferably the enzymes, essential oils mentioned in WO 2005/123101, preferably the insect repellents mentioned in US 2008/0070825, preferably the fibres, film formers, fixing agents, foam forming agents, foam stabilisers, substances for preventing foaming, foam boosters, fungicides, gelling agents and gel-forming agents mentioned in WO 2005/123101, preferably the hair care agents, hair shaping agents, hair smoothing agents, moisture regulators (moisture-dispensing, moisturising and/or moisture-containing substances) mentioned in WO 2005/123101, preferably the osmolytes mentioned in WO 2005/123101, preferably the compatible solutes mentioned in WO 2005/123101, preferably the bleaching agents, strengthening agents, stain-removing agents, optically brightening agents, impregnating agents, dirt-repellent agents, friction-reducing agents, lubricating agents, moisture creams, ointments, opacifying agents, plasticising agents, covering agents, polishes, brighteners, polymers mentioned in WO 01/76572 and WO 02/15686, preferably the powders, proteins and protein hydrolysates mentioned in WO 2008/046676, preferably the lipid regulating agents, exfoliating agents, skin-calming agents, skin cleaning agents, skin care agents, skin repair agents, preferably containing cholesterol and/or fatty acids and/or ceramides and/or pseudo-ceramides mentioned in WO 2005/123101 and WO 2008/046676, in this case preferably the skin brightening agents mentioned in WO 2006/053912, preferably the skin protecting agents, skin softening agents, skin cooling agents mentioned in WO 2007/110415, preferably the skin warming agents mentioned in WO 2005/123101, preferably the stabilisers, UV-absorbing agents and UV filters mentioned in WO 2005/123101, preferably the benzylidene-betadicarbonyl compounds mentioned in WO 2005/123101, preferably the alpha-benzoyl cinnamic acid nitriles mentioned in WO 2005/107692, preferably the AhR-receptor antagonists mentioned in WO 2006/015954, preferably the detergents, fabric conditioners, suspending agents, skin tanning agents mentioned in WO2007/128723 and WO 2007/060256, preferably the thickeners, vitamins mentioned in WO 2006/045760, preferably the oils, waxes and fats mentioned in WO 2005/123101, preferably the phospholipids mentioned in WO 2005/123101, preferably the fatty acids (saturated fatty acids, singly or multiply unsaturated fatty acids, α-hydroxy acids, polyhydroxy fatty acids) mentioned in WO 2005/123101, preferably the liquefiers, dyes and colour-protecting agents and pigments mentioned in WO 2005/123101, preferably the anti-corrosives, flavours and flavour additives and further additional fragrance substances mentioned in WO 2005/123101, preferably the further fragrance substances listed in S. Arctander, Perfume and Flavor Chemicals, Eigenverlag, Montclair, N.J., 1969 and Surburg, Panten, Common Fragrance and Flavor Materials, 5^{th} Edition, Wiley-VCH, Weinheim 2006, in particular the further fragrance substances explicitly mentioned in US 2008/0070825, which are not already part of constituents (a) and (b) and optionally (c) and/or (d) of a fragrance substance mixture according to the invention or a perfume oil according to the invention, alcohols and polyols, preferably the surfactants mentioned in WO 2005/123101, preferably the animal extracts, yeast extracts, extracts of algae or micro-algae, electrolytes, liquefiers, organic solvents mentioned in WO 2005/123101, preferably those mentioned in WO 2005/123101, or silicones and silicone derivatives, preferably those mentioned in WO 2008/046676.

Furthermore, fragrance substance mixtures according to the invention, in particular perfume oils, can be adsorbed on a carrier material, which ensures both a fine distribution of the fragrance substances contained therein in the product and also a controlled release during application. Carriers of this type may be porous inorganic materials such as light sulphate, silica gels, zeolites, plasters, clays, clay granulates, gas concrete etc. or organic materials such as timbers, cellulose-based substances, sugar, dextrins (for example maltodextrin) or plastics materials such as PVC, polyvinyl acetates or polyurethanes. The combination of the composition according to the invention and carrier substance is an exemplary article according to the invention.

Fragrance substance mixtures according to the invention, in particular perfume oils, may also be present microencapsulated, spray dried, as inclusion complexes or as extrusion products (i.e. products according to the invention) and be added in this form, for example, to a product to be perfumed.

Optionally, the properties of the compositions modified in this way can be further optimised by so-called “coating” with suitable materials with a view to a more targeted fragrance release, for which purpose, wax-like plastics materials, such as, for example, polyvinyl alcohol, are preferably used. The resulting products are in turn products according to the invention.

The microencapsulation of the fragrance substance mixtures according to the invention, preferably the perfume oils according to the invention, may, for example, take place by the so-called coacervation method with the aid of capsule materials, for example made of polyurethane-like substances or soft gelatines. The spray-dried fragrance or aromatic substance compositions may, for example, be produced by spray drying an emulsion or dispersion containing the fragrance substance mixture according to the invention, preferably a perfume oil, whereupon modified starches, proteins, dextrin and vegetable rubbers can be used as carrier materials. Inclusion complexes may, for example, be produced by introducing dispersions of the fragrance substance mixture according to the invention, preferably a perfume oil according to the invention, and cyclodextrins or urea derivatives into a suitable solvent, for example water. Extrusion products may be obtained by melting a fragrance substance mixture according to the invention, preferably a perfume oil according to the invention, with a suitable wax-like substance and by extrusion with subsequent solidification, optionally in a suitable solvent, for example isopropanol.

Fragrance substance mixtures according to the invention, preferably perfume oils according to the invention, can preferably be used as such, in concentrated form, in solutions or in the modified form described above to produce perfumed products according to the invention.

Preferred perfumed products according to the invention contain a fragrance substance mixture according to the invention, preferably a perfume oil according to the invention, in a sensorily effective quantity.

The total mass of the compounds (A-1) and (A-2) (constituent (a)) in a fragrance substance mixture according to the invention (as defined above), preferably in one of the embodiments designated as preferred, or in a perfumed product (as defined above), preferably in one of the embodiments designated as preferred, is preferably in the range from 0.25 to 10% by weight, preferably in the range from 0.5 to 8% by weight, particularly preferably in the range from 0.75 to 7.5% by weight, particularly preferably in the range from 1 to 6% by weight, in each case based on the total mass of all the fragrance substances contained in the fragrance substance mixture or the perfumed product.

In this case, the total mass of all the fragrance substances contained in the fragrance substance mixture or the perfumed product preferably relates to the total mass of all the fragrance substances with a molar mass of less than or equal to 300 g/mol, preferably with a molar mass of less than or equal to 290 g/mol, particularly preferably with a molar mass of less than or equal to 280 g/mol.

The above described olfactory effects, in particular the diverse modifying and masking effects, occur, in particular, in the quantity fractions given as preferred above or as particularly preferred. The modifying effects recede into the background and are no longer observed to the desired extent if the total mass of compounds (A-1) and (A-2) based on the total mass of all the fragrance substances contained in a fragrance substance mixture or a perfumed product, is selected to be too high (i.e. significantly above the quantity ranges given above) as, in a case such as this, the odour particular to the compounds (A-1) and (A-2) come too strongly to the fore and the desired modifying effects are concealed or less strongly perceived with an increasing exceeding of the quantity fractions given above.

The fraction of a fragrance substance mixture according to the invention, preferably a perfume oil according to the invention, in perfumed products according to the invention is typically 0.01 to 10% by weight, preferably 0.1 to 5% by weight and particularly preferably 0.25 to 3% by weight, in each case based on the total mass of the perfumed product.

Preferred perfumed products according to the invention are washing and cleaning agents, hygiene or care products, in particular in the area of body and hair care, cosmetics and the household.

Preferred perfumed products according to the invention are selected from the following list: perfume extracts, eau de parfums, eau de toilettes, after-shaves, eau de cologne, pre-shave products, splash colognes and perfumed refresher tissues, acid, alkaline and neutral cleaning agents, such as, for example, floor cleaners, window glass cleaners, dishwasher detergents, bath and sanitary cleaners, scouring milk, solid and liquid WC cleaners, powdery and foam carpet cleaners, textile fresheners, ironing aids, liquid washing agents, powder-like washing agents, laundry pre-treatment agents such as bleaching agents, soaking agents and stain removers, laundry conditioners, washing soaps, washing tablets, disinfectants, surface disinfection agents and air improvers in liquid, gel-like form or in a form applied on a solid carrier, aerosol sprays, waxes and polishes such as furniture polishes, floor waxes, shoe creams, body care agents, such as, for example, solid and liquid soaps, shower gels, shampoos, shaving soaps, shaving foams, bath oils, cosmetic emulsions of the oil-in-water, of the water-in-oil and of the water-in-oil-in-water type such as, for example, skin creams and lotions, face creams and lotions, sun protection creams and lotions, after sun creams and lotions, hand creams and lotions, foot creams and lotions, depilatory creams and lotions, after-shave creams and lotions, skin tanning creams and lotions, skin brightening creams and lotions, hair care products, such as, for example, hairsprays, hair gels, hair setting lotions, hair rinses, permanent and semi-permanent hair dyes, hair shaping means such as cold waves and hair smoothing agents, hair tonics, hair creams and lotions, deodorants and antiperspirants such as, for example, underarm sprays, roll-ons, deodorant sticks, deodorant creams or products of decorative cosmetics, such as, for example eye shadows, nail varnishes, make ups, lipsticks, mascara as well as for candles, lamp oils, joss sticks, insecticides and repellents.

Particularly preferred perfumed products according to the invention are selected from the following list:
eau de parfums, eau de toilettes, after shaves, eau de colognes, pre-shave products, splash colognes;
acid, alkaline and neutral cleaning agents, in particular in the household area, preferably floor cleaners, window glass cleaners, dishwasher detergents, bath and sanitary cleaners, scouring milk, solid and liquid WC cleaners, powdery and foam carpet cleaners, liquid washing agents, powdery washing agents, laundry conditioners, surface disinfection agents, in particular for hard surfaces (hard surface cleaner);
body care agents, preferably solid and liquid soaps, shower gels, shampoos, shaving soaps, shaving foams;
cosmetic emulsions of the oil-in-water, water-in-oil and water-in-oil-in-water type, preferably skin creams and lotions, face creams and lotions, sun protection creams and lotions, after sun creams and lotions, hand creams and lotions, foot creams and lotions, depilatory creams and lotions, after shave creams and lotions, skin tanning creams and lotions, skin brightening creams and lotions;
hair care products, preferably hair sprays, hair gels, hair setting lotions, hair rinses, permanent and semi-permanent hair dyes, hair tonics, hair creams and lotions;
deodorants and antiperspirants, preferably armpit sprays, roll-ons (preferably as an alcoholic or non-alcoholic solution, as a gel or (micro)emulsion), deodorant sticks, deodorant creams.

The following examples explain the invention; if nothing else is stated, the fractions and percentages relate to the weight. In the following exemplary perfume oils according to the invention and perfumed products, the above-described olfactory effects were observed.

### EXAMPLES

Abbreviations used:

Dipropylene glycol (DPG), diethyl phthalate (DEP), triethyl citrate (TEC), isopropyl myristate (IPM); nat=natural, macrocyclic musk fragrance substance (constituent (b) of the fragrance substance mixture according to the invention (MACRO)); polycyclic musk fragrance substance (constituent (b) of the fragrance substance mixture according to the invention (POLY)); alicyclic musk fragrance substance (constituent (b) of the fragrance substance mixture according to the invention (ALICYC));
HEDIONE® HC/30 contains at least 30% by weight of the cis-isomer and just 70% by weight of the trans-isomer of methyl dihydrojasmonate.

For explanations of the product names of the fragrance substances see, for example, S. Arctander, Perfume and Flavor Materials, Vol. I and II, Montclair, N.J., 1969, self-published or H. Surburg, J. Panten “Common Fragrance and Flavor Materials”, 5^{th} ed., Wiley-VCH, Weinheim 2006.

The following mixtures used (Mix1), (Mix2) and (Mix3) of the compounds (A1) and (A2) to be used according to the invention were produced analogously to the methods described in EP 1 178 105, the isomeric mixtures having been obtained either by means of fractionation of the corresponding reaction mixtures or the compounds (A1) and (A2) were initially produced in as pure a form as possible and then the respective weight ratio of (A1) to (A2) were adjusted in a targeted and precise manner by mixing the corresponding quantity fractions of the two pure compounds (A1) and (A2).

The mixture (Mix1) used below contained the compounds (A1) and (A2) in the weight ratio 3.6:1. The quantity of the two compounds (A1) and (A2) was 96.1% by weight, based on the total weight of (Mix1).

The mixture (Mix2) used below contained the compounds (A1) and (A2) in the weight ratio 4.5:1. The quantity of the two compounds (A1) and (A2) was 97.5% by weight, based on the total weight of (Mix2).

The mixture (Mix3) used below contained the compounds (A1) and (A2) in the weight ratio 5.8:1. The quantity of the two compounds (A1) and (A2) was 98.2% by weight, based on the total weight of (Mix3).

### Perfume Oil Example P1

| | Ref-P1 | P1 | P1a |
|---|---|---|---|
| AMBRETTOLIDE (MACRO) | 10.00 | 10.00 | 10.00 |
| AMBROXIDE 10% in IPM | 10.00 | 10.00 | 10.00 |
| BENZYL ACETATE | 20.00 | 20.00 | 20.00 |
| BENZYL SALICYLATE | 15.00 | 15.00 | 15.00 |
| BERGAMOT OIL, bergapten-free | 60.00 | 60.00 | 60.00 |
| CALONE ® 1951 10% in DPG | 15.00 | 15.00 | 15.00 |
| COUMARIN | 5.00 | 5.00 | 5.00 |
| CYCLOGALBANATE ® 10% in DPG | 10.00 | 10.00 | 10.00 |
| ALPHA-DAMASCONE 1% in DPG | 20.00 | 20.00 | 20.00 |
| DIHYDROMYRCENOL | 10.00 | 10.00 | 10.00 |
| ETHYL LINALOOL | 75.00 | 75.00 | 75.00 |
| ETHYL LINALYL ACETATE | 50.00 | 50.00 | 50.00 |
| ETHYL MALTOL 1% in DEP | 10.00 | 10.00 | 10.00 |
| ETHYLENE BRASSYLATE (MACRO) | 90.00 | 90.00 | 90.00 |
| FLOROSA | 40.00 | 40.00 | 40.00 |
| GERANYL ACETATE | 10.00 | 10.00 | 10.00 |
| HEDIONE ® HC/30 | 35.00 | 35.00 | 35.00 |
| HEDIONE ® | 210.00 | 210.00 | 210.00 |
| HELIONAL ® | 15.00 | 15.00 | 15.00 |
| HELVETOLIDE ® (ALICYC) | 30.00 | 30.00 | 30.00 |
| HEXENYL SALICYLATE CIS-3 | 20.00 | 20.00 | 20.00 |
| ISO E SUPER ® | 40.00 | 40.00 | 40.00 |
| LEAFOVERT ® 10% in DEP | 10.00 | 10.00 | 10.00 |
| LILIAL ® | 80.00 | 80.00 | 80.00 |
| LYRAL ® | 20.00 | 20.00 | 20.00 |
| MANDARIN OIL | 10.00 | 10.00 | 10.00 |
| STYRALYL ACETATE | 5.00 | 5.00 | 5.00 |
| SYMROSE ® | 15.00 | 15.00 | 15.00 |
| VANILLIN 10% in DEP | 20.00 | 20.00 | 20.00 |
| DIPROPYLENE GLYCOL (DPG) | 40.00 | 10.00 | 10.00 |
| MIXTURE (Mix2) | — | 30.00 | — |
| MIXTURE (Mix1) | — | — | 30.00 |
| TOTAL: | 1,000.00 | 1,000.00 | 1,000.00 |

### Perfume Oil Example P2

| | Ref-P2 | P2 | P2a |
|---|---|---|---|
| AMAROCIT ® | 10.00 | 10.00 | 10.00 |
| AMBROCENIDE ® 10% in DPG | 5.00 | 5.00 | 5.00 |
| AMBROXIDE | 15.00 | 15.00 | 15.00 |
| AURELIONE ® (7/8- | 70.00 | 70.00 | 70.00 |
| Cyclohexadecenone) (MACRO) | | | |
| BERGAMOT OIL, bergapten-free | 90.00 | 90.00 | 90.00 |
| CALONE ® 1951 10% in DPG | 20.00 | 20.00 | 20.00 |
| CARAWAY OIL | 10.00 | 10.00 | 10.00 |
| CITRAL | 20.00 | 20.00 | 20.00 |
| COUMARIN | 10.00 | 10.00 | 10.00 |
| ALPHA-DAMASCONE 1% in DPG | 15.00 | 15.00 | 15.00 |
| DIHYDROMYRCENOL | 70.00 | 70.00 | 70.00 |
| ESTRAGON OIL | 10.00 | 10.00 | 10.00 |
| ETHYL LINALOOL | 100.00 | 100.00 | 100.00 |
| ETHYL LINALYLACETATE | 90.00 | 90.00 | 90.00 |
| EUGENOL | 10.00 | 10.00 | 10.00 |
| EVERNYL ® | 5.00 | 5.00 | 5.00 |
| FRUCTATE ® | 5.00 | 5.00 | 5.00 |
| GERANIUM OIL | 5.00 | 5.00 | 5.00 |
| HEDIONE ® HC/30 | 100.00 | 100.00 | 100.00 |
| HELIONAL ® | 10.00 | 10.00 | 10.00 |
| INDOLE 10% in DPG | 5.00 | 5.00 | 5.00 |
| ISO E SUPER ® | 100.00 | 100.00 | 100.00 |
| KEPHALIS ® | 5.00 | 5.00 | 5.00 |
| LAVENDER OIL | 40.00 | 40.00 | 40.00 |
| CITRUS OIL | 80.00 | 80.00 | 80.00 |
| LILIAL ® | 30.00 | 30.00 | 30.00 |
| MANDARIN OIL | 20.00 | 20.00 | 20.00 |
| MUSCENONE (MACRO) | 5.00 | 5.00 | 5.00 |
| SANDRANOL ® | 10.00 | 10.00 | 10.00 |
| VANILLIN 10% in DPG | 5.00 | 5.00 | 5.00 |
| DIPROPYLENE GLYCOL | 30.00 | 10.00 | 10.00 |
| MIXTURE (Mix2) | — | 20.00 | — |
| MIXTURE (Mix3) | — | — | 20.00 |
| TOTAL: | 1,000.00 | 1,000.00 | 1,000.00 |

### Perfume Oil Example P3

| | Ref-P3 | P3 | P3a |
|---|---|---|---|
| ALDEHYDE C10 (n-decanal) 10% in DPG | 20.00 | 20.00 | 20.00 |
| ALDEHYDE C11 (n-undecanal) 10% in | 5.00 | 5.00 | 5.00 |
| DPG | | | |
| ALDEHYDE C12 (n-dodecanal) 10% in | 15.00 | 15.00 | 15.00 |
| DPG | | | |
| AMBRETTOLIDE (MACRO) | 5.00 | 5.00 | 5.00 |
| AMBROCENIDE ® 1% in DPG | 20.00 | 20.00 | 20.00 |
| AURELIONE ® (MACRO) | 30.00 | 30.00 | 30.00 |
| BENZYL ACETATE | 30.00 | 30.00 | 30.00 |
| CITRONELLOL | 15.00 | 15.00 | 15.00 |
| ETHYL VANILLIN 1% in DPG | 20.00 | 20.00 | 20.00 |
| ETHYLENE BRASSYLATE (MACRO) | 70.00 | 70.00 | 70.00 |
| FRUCTATE ® 10% in DPG | 20.00 | 20.00 | 20.00 |
| GERANYL ACETATE | 10.00 | 10.00 | 10.00 |
| GLOBALIDE ® (MACRO) | 30.00 | 30.00 | 30.00 |
| HEDIONE ® | 30.00 | 30.00 | 30.00 |
| ALPHA-HEXYLCINNAMALDEHYDE | 90.00 | 90.00 | 90.00 |
| INDOLE 10% in DPG | 5.00 | 5.00 | 5.00 |
| ISO E SUPER ® | 120.00 | 120.00 | 120.00 |
| KEPHALIS ® | 5.00 | 5.00 | 5.00 |
| LINALOOL | 150.00 | 150.00 | 150.00 |
| LINALYL ACETATE | 60.00 | 60.00 | 60.00 |
| BETA-METHYLNAPHTHYL KETONE | 5.00 | 5.00 | 5.00 |
| NEROLIDOL | 20.00 | 20.00 | 20.00 |
| NEROLIONE 10% in DPG | 20.00 | 20.00 | 20.00 |
| BRAZILIAN ORANGE OIL | 100.00 | 100.00 | 100.00 |
| PHENYLETHYL ACETATE | 5.00 | 5.00 | 5.00 |
| PHENYLETHYL ALCOHOL | 30.00 | 30.00 | 30.00 |
| TERPINEOL | 20.00 | 20.00 | 20.00 |
| DIPROPYLENE GLYCOL | 50.00 | 20.00 | 20.00 |
| MIXTURE (Mix1) | — | 30.00 | — |
| MIXTURE (Mix2) | — | — | 30.00 |
| TOTAL: | 1,000.00 | 1,000.00 | 1,000.00 |

### Perfume Oil Example P4

| | Ref-P4 | P4 | P4a |
|---|---|---|---|
| ALLYLAMYL GLYCOLATE | 10.00 | 10.00 | 10.00 |
| BERGAMOT OIL | 100.00 | 100.00 | 100.00 |
| CALONE ® 1951 10% in DPG | 30.00 | 30.00 | 30.00 |
| CASSIS BASE | 10.00 | 10.00 | 10.00 |
| CLOVE OIL | 5.00 | 5.00 | 5.00 |
| ALPHA-DAMASCONE 1% in DPG | 20.00 | 20.00 | 20.00 |
| DIHYDROMYRCENOL | 60.00 | 60.00 | 60.00 |
| ETHYLENE BRASSYLATE | 20.00 | 20.00 | 20.00 |
| (MACRO) | | | |
| FARENAL ® 1% in DPG | 20.00 | 20.00 | 20.00 |
| FLORALOZONE ® | 10.00 | 10.00 | 10.00 |
| GLOBALIDE ® (MACRO) | 20.00 | 20.00 | 20.00 |
| HEDIONE ® HC/30 | 10.00 | 10.00 | 10.00 |
| HEDIONE ® | 200.00 | 200.00 | 200.00 |
| ALPHA- | 100.00 | 100.00 | 100.00 |
| HEXYLCINNAMALDEHYDE | | | |
| HEXYL SALICYLATE | 20.00 | 20.00 | 20.00 |
| ISO E SUPER ® | 70.00 | 70.00 | 70.00 |
| KEPHALIS | 5.00 | 5.00 | 5.00 |
| LAVANDER OIL GROSSO NAT. | 20.00 | 20.00 | 20.00 |
| LEAFOVERT ® 10% in DPG | 20.00 | 20.00 | 20.00 |
| LIGUSTRAL 10% in DPG | 10.00 | 10.00 | 10.00 |
| LILIAL ® | 45.00 | 45.00 | 45.00 |
| LINALOOL | 60.00 | 60.00 | 60.00 |
| LINALYL ACETATE | 50.00 | 50.00 | 50.00 |
| MANDARIN ALDEHYDE 10% in | 5.00 | 5.00 | 5.00 |
| TEC | | | |
| BRAZILIAN ORANGE OIL | 25.00 | 25.00 | 25.00 |
| POLYSANTOL ® | 5.00 | 5.00 | 5.00 |
| TERPINEOL | 20.00 | 20.00 | 20.00 |
| DIPROPYLENE GLYCOL | 30.00 | — | — |
| MIXTURE (Mix1) | — | 30.00 | — |
| MIXTURE (Mix3) | — | — | 30.00 |
| TOTAL: | 1,000.00 | 1,000.00 | 1,000.00 |

### Perfume Oil Example P5

| | Ref-P5 | P5 | P5a |
|---|---|---|---|
| Florazone | 15.00 | 15.00 | 15.00 |
| Cyclogalbanate ® | 15.00 | 15.00 | 15.00 |
| Bergamot oil | 135.00 | 135.00 | 135.00 |
| Mandarin oil | 50.00 | 50.00 | 50.00 |
| Claritone ® | 100.00 | 100.00 | 100.00 |
| Oxanthia 5% in DPG | 5.00 | 5.00 | 5.00 |
| Amarocit ® | 10.00 | 10.00 | 10.00 |
| Peppermint oil | 5.00 | 5.00 | 5.00 |
| Artemosia oil | 5.00 | 5.00 | 5.00 |
| Mace oil | 5.00 | 5.00 | 5.00 |
| Lilial ® | 50.00 | 50.00 | 50.00 |
| Calone ® 1951 10% in DPG | 25.00 | 25.00 | 25.00 |
| Geranium oil | 10.00 | 10.00 | 10.00 |
| Hedione ® | 240.00 | 240.00 | 240.00 |
| Hexenyl salicylate cis-3 | 15.00 | 15.00 | 15.00 |
| Parmanyl ® | 5.00 | 5.00 | 5.00 |
| Beta-Ionone | 25.00 | 25.00 | 25.00 |
| Cedarwood oil | 30.00 | 30.00 | 30.00 |
| Iso E Super ® | 80.00 | 80.00 | 80.00 |
| Vetival ® | 40.00 | 40.00 | 40.00 |
| Vetikolacetat ® | 5.00 | 5.00 | 5.00 |
| Sandranol ® | 75.00 | 75.00 | 75.00 |
| Ysamber ® K | 10.00 | 10.00 | 10.00 |
| Globalide ® (MACRO) | 25.00 | 25.00 | 25.00 |
| Dipropylene glycol | 20.00 | 15.00 | 15.00 |
| MIXTURE (Mixl ) | — | 5.00 | — |
| MIXTURE (Mix2) | — | — | 5.00 |
| TOTAL: | 1,000.00 | 1,000.00 | 1,000.00 |

### Perfume Oil Example P6

| | Ref-P6 | P6 | P6a |
|---|---|---|---|
| Leafovert ® 10% in IPM | 10.00 | 10.00 | 10.00 |
| Bergamot oil | 50.00 | 50.00 | 50.00 |
| Orange oil | 40.00 | 40.00 | 40.00 |
| Oxanthia 10% in DPG | 5.00 | 5.00 | 5.00 |
| Black pepper oil 10% in DPG | 20.00 | 20.00 | 20.00 |
| Camphor 10% in DPG | 5.00 | 5.00 | 5.00 |
| Gamma-decalactone 10% in DPG | 15.00 | 15.00 | 15.00 |
| Gamma-undecalactone 10% in DPG | 5.00 | 5.00 | 5.00 |
| Lilial ® | 60.00 | 60.00 | 60.00 |
| Helional | 25.00 | 25.00 | 25.00 |
| Linalool | 80.00 | 80.00 | 80.00 |
| Geranium oil 10% in DPG | 10.00 | 10.00 | 10.00 |
| Citronellol | 20.00 | 20.00 | 20.00 |
| Geraniol | 15.00 | 15.00 | 15.00 |
| Isodamascone ® 10% in DPG | 20.00 | 20.00 | 20.00 |
| Hedione ® | 120.00 | 120.00 | 120.00 |
| Alpha-Hexylcinnamaldehyde | 40.00 | 40.00 | 40.00 |
| Vanillin | 15.00 | 15.00 | 15.00 |
| Coumarone ® | 5.00 | 5.00 | 5.00 |
| Iso E Super ® | 240.00 | 240.00 | 240.00 |
| Patchouli oil nat. | 25.00 | 25.00 | 25.00 |
| Sandalore | 10.00 | 10.00 | 10.00 |
| Labdanum Absolute 10% in DPG | 5.00 | 5.00 | 5.00 |
| Ysamber ® K | 5.00 | 5.00 | 5.00 |
| Macrolide ® Supra (MACRO) | 25.00 | 25.00 | 25.00 |
| Globalide ® (MACRO) | 45.00 | 45.00 | 45.00 |
| Cyclohexadecanone (MACRO) | 15.00 | 15.00 | 15.00 |
| Ethylene brassylate (MACRO) | 45.00 | 45.00 | 45.00 |
| Indole 10% in DPG | 5.00 | 5.00 | 5.00 |
| Dipropylene glycol | 20.00 | 10.00 | 10.00 |
| MIXTURE (Mixl ) | — | 10.00 | — |
| MIXTURE (Mix2) | — | — | 10.00 |
| TOTAL: | 1,000.00 | 1,000.00 | 1,000.00 |

### Perfume Oil Example P7

| | Ref-P7 | P7 | P7a |
|---|---|---|---|
| Styrallyl acetate | 10.00 | 10.00 | 10.00 |
| Linalyl acetate | 40.00 | 40.00 | 40.00 |
| Mandaril 10% in DPG | 5.00 | 5.00 | 5.00 |
| Orange oil | 60.00 | 60.00 | 60.00 |
| Oxanthia 5% in DPG | 5.00 | 5.00 | 5.00 |
| Gamma-decalactone | 5.00 | 5.00 | 5.00 |
| Ethyl-3-methyl-3-phenylglycidate | 5.00 | 5.00 | 5.00 |
| Frambinon ® | 5.00 | 5.00 | 5.00 |
| Raspberry base | 5.00 | 5.00 | 5.00 |
| Lilial ® | 80.00 | 80.00 | 80.00 |
| Calone ® 1951 10% in DPG | 20.00 | 20.00 | 20.00 |
| Majantol ® | 80.00 | 80.00 | 80.00 |
| Linalool | 60.00 | 60.00 | 60.00 |
| Phenylethyl alcohol | 20.00 | 20.00 | 20.00 |
| Citronellol | 20.00 | 20.00 | 20.00 |
| Geraniol | 5.00 | 5.00 | 5.00 |
| Benzyl acetate | 30.00 | 30.00 | 30.00 |
| Hedione ® | 150.00 | 150.00 | 150.00 |
| Alpha-Hexylcinnamaldehyde | 150.00 | 150.00 | 150.00 |
| Benzyl salicylate | 60.00 | 60.00 | 60.00 |
| Amyl salicylate (n- and iso-) | 40.00 | 40.00 | 40.00 |
| Beta-Ionone | 20.00 | 20.00 | 20.00 |
| Eugenol | 5.00 | 5.00 | 5.00 |
| Ethyl vanillin 10% in DPG | 10.00 | 10.00 | 10.00 |
| Cinnamyl alcohol | 5.00 | 5.00 | 5.00 |
| Iso E Super ® | 40.00 | 40.00 | 40.00 |
| Galaxolide ® 50% in DEP | 25.00 | 25.00 | 25.00 |
| (4,6,6,7,8,8-Hexamethyl- | | | |
| 1,3,4,6,7,8- | | | |
| hexahydro- | | | |
| cyclopenta[g]benzopyrane) (POLY) | | | |
| Globalide ® (MACRO) | 20.00 | 20.00 | 20.00 |
| Dipropylene glycol | 20.00 | 15.00 | 15.00 |
| MIXTURE (Mix2) | — | 5.00 | — |
| MIXTURE (Mix3) | — | — | 5.00 |
| TOTAL: | 1,000.00 | 1,000.00 | 1,000.00 |

### Perfume Oil Example P8

| | Ref-P8 | P8 | P8a |
|---|---|---|---|
| Limonenal 10% in DPG | 6.00 | 6.00 | 6.00 |
| Profarnesal 10% in DPG | 4.00 | 4.00 | 4.00 |
| Hexenyl butyrate cis-3 | 2.00 | 2.00 | 2.00 |
| Mintonat ® | 3.00 | 3.00 | 3.00 |
| Linalyl acetate | 120.00 | 120.00 | 120.00 |
| Citrus oil | 20.00 | 20.00 | 20.00 |
| Citrylal | 2.00 | 2.00 | 2.00 |
| Gamma-nonalactone | 15.00 | 15.00 | 15.00 |
| Frambinon ® | 2.00 | 2.00 | 2.00 |
| Raspberry base | 20.00 | 20.00 | 20.00 |
| Filbertone 1% in DPG | 1.00 | 1.00 | 1.00 |
| Acetylmethylcarbinol, nat. 10% in DPG | 7.00 | 7.00 | 7.00 |
| Lilax Soft | 20.00 | 20.00 | 20.00 |
| Linalool | 30.00 | 30.00 | 30.00 |
| Terpinenol | 80.00 | 80.00 | 80.00 |
| Phenylethyl alcohol | 20.00 | 20.00 | 20.00 |
| Geraniol | 40.00 | 40.00 | 40.00 |
| Nerol | 70.00 | 70.00 | 70.00 |
| Citronellol | 30.00 | 30.00 | 30.00 |
| Benzyl acetate | 10.00 | 10.00 | 10.00 |
| Alpha-Amylcinnamaldehyde | 60.00 | 60.00 | 60.00 |
| Hedione ® | 130.00 | 130.00 | 130.00 |
| Beta-Ionone | 10.00 | 10.00 | 10.00 |
| Agrumex ® HC | 80.00 | 80.00 | 80.00 |
| Oryclon ® | 68.00 | 68.00 | 68.00 |
| Cedarwood oil | 2.00 | 2.00 | 2.00 |
| Vertofix ® | 120.00 | 120.00 | 120.00 |
| Macrolide ® (MACRO) | 20.00 | 20.00 | 20.00 |
| Triethyl citrate | 8.00 | 5.00 | 5.00 |
| MIXTURE (Mix3) | — | 3.00 | — |
| MIXTURE (Mix1) | — | — | 3.00 |
| TOTAL: | 1,000.00 | 1,000.00 | 1,000.00 |

### Perfume Oil Example P9

| | Ref-P9 | P9 | P9a |
|---|---|---|---|
| AMAROCIT ® | 300.00 | 300.00 | 300.00 |
| AMBROXIDE | 150.00 | 150.00 | 150.00 |
| BENZYL SALICYLATE | 4,410.00 | 4,410.00 | 4,410.00 |
| CALONE ® 1951 | 150.00 | 150.00 | 150.00 |
| CLARY SAGE OIL | 60.00 | 60.00 | 60.00 |
| DAMASCONE ALPHA, 10% in DPG | 70.00 | 70.00 | 70.00 |
| EVERNYL, 10% in DPG | 150.00 | 150.00 | 150.00 |
| GLOBANONE ® | 1,200.00 | 1,200.00 | 1,200.00 |
| HELIONAL | 300.00 | 300.00 | 300.00 |
| HEXENYL SALICYLATE CIS-3 | 1,500.00 | 1,500.00 | 1,500.00 |
| ISOBUTYL CHINOLINE 1% in DPG | 150.00 | 150.00 | 150.00 |
| LILIAL ® | 450.00 | 450.00 | 450.00 |
| POLYSANTOL | 60.00 | 60.00 | 60.00 |
| SYMMARINE ® 1% in DPG 10% DPG | 50.00 | 50.00 | 50.00 |
| MIXTURE (Mix2) | — | 500.00 | — |
| MIXTURE (Mix3) | — | — | 500.00 |
| TOTAL: | 9,000.00 | 9,500.00 | 9,500.00 |

### Perfume Oil Example P10

| FLORALOZONE | Ref-P10 | P10 | P10a |
|---|---|---|---|
| ALLYLAMYL GLYCOLATE | 2.00 | 2.00 | 2.00 |
| CYCLOGALBANAT ® | 6.00 | 6.00 | 6.00 |
| MINTONATE | 4.00 | 4.00 | 4.00 |
| DIHYDROMYRCENOL | 60.00 | 60.00 | 60.00 |
| TERPINYL ACETATE | 120.00 | 120.00 | 120.00 |
| CITRUS OIL | 60.00 | 60.00 | 60.00 |
| ORANGE OIL, | 20.00 | 20.00 | 20.00 |
| ARTEMISIA OIL | 30.00 | 30.00 | 30.00 |
| BASIL OIL | 2.00 | 2.00 | 2.00 |
| BLACK PEPPER OIL | 1.00 | 1.00 | 1.00 |
| ALDEHYDE C14 so-called, 10% in | 4.00 | 4.00 | 4.00 |
| DPG | | | |
| FRUCTATE ® | 1.50 | 1.50 | 1.50 |
| PEACHOLIDE, 10% in DPG | 0.50 | 0.50 | 0.50 |
| MUGETANOL | 4.00 | 4.00 | 4.00 |
| LINALOOL | 40.00 | 40.00 | 40.00 |
| GERANIUM OIL | 5.00 | 5.00 | 5.00 |
| HEXYLCINNAMALDEHYDE ALPHA | 30.00 | 30.00 | 30.00 |
| ETHYL VANILLIN | 1.00 | 1.00 | 1.00 |
| VANILLIN | 3.00 | 3.00 | 3.00 |
| COUMARIN | 20.00 | 20.00 | 20.00 |
| AGRUMEX | 1.50 | 1.50 | 1.50 |
| CERARWOOD OIL | 20.00 | 20.00 | 20.00 |
| VERTOFIX | 40.00 | 40.00 | 40.00 |
| AMBERWOOD ® F | 15.00 | 15.00 | 15.00 |
| CEDRAMBER | 30.00 | 30.00 | 30.00 |
| ISO E SUPER ® | 180.00 | 180.00 | 180.00 |
| TRIMOFIX O | 10.00 | 10.00 | 10.00 |
| KOHINOOL | 10.00 | 10.00 | 10.00 |
| ISOLONGIFOLANOL | 5.00 | 5.00 | 5.00 |
| EBANOL | 5.00 | 5.00 | 5.00 |
| SANDALORE | 20.00 | 20.00 | 20.00 |
| SANDRANOL ® | 60.00 | 60.00 | 60.00 |
| GUAIAC WOOD OIL | 8.00 | 8.00 | 8.00 |
| AMBROCENIDE ® | 2.00 | 2.00 | 2.00 |
| AMBROXIDE | 2.00 | 2.00 | 2.00 |
| YSAMBER ® K | 18.00 | 18.00 | 18.00 |
| GLOBALIDE ® (MACRO) | 24.00 | 24.00 | 24.00 |
| AURELIONE ® (MACRO) | 16.00 | 16.00 | 16.00 |
| DPG | 18.00 | — | — |
| MIXTURE (Mix2) | — | 18.00 | — |
| MIXTURE (Mix3) | — | — | 18.00 |
| TOTAL: | 900.00 | 900.00 | 900.00 |

### Perfume Oil Example P11

| | P11 | P11a |
|---|---|---|
| Acetophenone, 10% in DPG | 10.00 | 10.00 |
| n-undecanal | 5.00 | 5.00 |
| Aldehyde C14 so-called (peach aldehyde) | 15.00 | 15.00 |
| Allylamyl glycolate, 10% in DPG | 20.00 | 20.00 |
| Amyl salicylate | 25.00 | 25.00 |
| Benzyl acetate | 60.00 | 60.00 |
| Citronellol | 80.00 | 80.00 |
| D-Limonene | 50.00 | 50.00 |
| Decenol trans-9 | 15.00 | 15.00 |
| Dihydromyrcenol | 50.00 | 50.00 |
| Dimethyl benzyl carbinyl acetate | 30.00 | 30.00 |
| Diphenyl oxide | 5.00 | 5.00 |
| Eucalyptol | 10.00 | 10.00 |
| Geraniol | 40.00 | 40.00 |
| Nerol | 20.00 | 20.00 |
| Geranium oil | 15.00 | 15.00 |
| Hexenol cis-3, 10% in DPG | 5.00 | 5.00 |
| Hexenyl salicylate cis-3 | 20.00 | 20.00 |
| Indol, 10% in DPG | 10.00 | 10.00 |
| Alpha-Ionone | 15.00 | 15.00 |
| Isolongifolanol | 30.00 | 20.00 |
| Lilial (2-Methyl-3-(4-tert-butyl-phenyl)propanal) | 55.00 | 55.00 |
| Linalool | 40.00 | 40.00 |
| Methylphenyl acetate | 10.00 | 10.00 |
| Phenylethyl alcohol | 180.00 | 180.00 |
| Styralyl acetate | 20.00 | 20.00 |
| Terpineol | 30.00 | 30.00 |
| Tetrahydro linalool | 40.00 | 50.00 |
| Cinnamyl alcohol | 10.00 | 10.00 |
| Macrolide (15-Pentadecanolide) (MACRO) | 115.00 | 100.00 |
| 5-Cyclohexadecen-1-one (MACRO) | — | 25.00 |
| MIXTURE (Mix2) | 30.00 | — |
| MIXTURE (Mix3) | — | 45.00 |
| Total: | 1,060.00 | 1,085.00 |

### Perfume Oil Example P12

| | P12 | P12a |
|---|---|---|
| Benzyl acetate | 60.00 | 60.00 |
| Citronellyl acetate | 60.00 | 60.00 |
| Cyclamenaldehyde (2-Methyl-3-(4- | 20.00 | 20.00 |
| isopropylphenyl)propanal | | |
| Dipropylene glycol | 100.00 | 60.00 |
| Ethyl linalool | 40.00 | 40.00 |
| Florol (2-Isobutyl-4-methyltetrahydro-2H-pyran-4-ol) | 30.00 | 30.00 |
| Globanone [(E/Z)-8-Cyclohexadecen-1-one] | 180.00 | 180.00 |
| (MACRO) | | |
| Hedione (Methyl dihydrojasmonate) | 140.00 | 140.00 |
| Hexenyl salicylate, cis-3 | 10.00 | 10.00 |
| Vertocitral (2,4-dimethyl-3- | 5.00 | 5.00 |
| cyclohexencarboxaldehyde) | | |
| Hydratropaaldehyde, 10% in DPG | 5.00 | 5.00 |
| Isodamascone (1-(2,4,4-Trimethyl-2-cyclohexen-1- | 5.00 | 5.00 |
| yl)-2-buten-1-one), 10% in DPG | | |
| Isomuscone (Cyclohexadecanone) (MACRO) | 40.00 | 40.00 |
| Jacinthaflor (2-Methyl-4-phenyl-1,3-dioxolane) | 10.00 | 10.00 |
| Cis-jasmone, 10% in DPG | 20.00 | 20.00 |
| Linalool | 75.00 | 75.00 |
| Linalyl acetate | 30.00 | 30.00 |
| Methyl benzoate, 10% in DPG | 25.00 | 25.00 |
| para-Methyl cresol, 10% in DPG | 10.00 | 10.00 |
| Nerol | 40.00 | 40.00 |
| Phenylpropylaldehyde | 5.00 | 5.00 |
| 2-Phenylethyl alcohol | 80.00 | 80.00 |
| Tetrahydro geraniol | 30.00 | 20.00 |
| 2,2-Dimethyl-3-cyclohexyl-1-propanol | 10.00 | 20.00 |
| MIXTURE (Mix2) | 20.00 | 40.00 |
| Total: | 1,050.00 | 1,030.00 |

The perfume oils according to the invention P1, P2, P3, P4, P5, P6, P7, P8, P9, P10, P11 and P12 from the above perfume oil examples P1 to P12 were in each case incorporated separately into the following formulations.

The olfactory effects observed in the respective perfume oil were in each case also observed in the following formulations.

### FORMULATION EXAMPLES

### Example F1

### Washing Powder

| | | | % by | % by |
|---|---|---|---|---|
| Material | Chemical Name | Function | weight | weight |
| Sodium metasilicate | Sodium metasilicate | | Ad 100 | Ad 100 |
| pentahydrate | pentahydrate | | | |
| Sodium hydrogen | Sodium hydrogen | Alkali | 15.0 | 15.0 |
| carbonate | carbonate | | | |
| Sodium percarbonate | Sodium carbonate | Bleaching | 15.0 | 15.0 |
| | peroxyhydrate | agent | | |
| Peractive AC | TAED/Na Carboxy- | Activator | 5.00 | 5.00 |
| Blue | methylcellulose | | | |
| Genapol OA-080 | Oxoalkohol C14-15, | Non-ionic | 3.00 | 3.00 |
| | 8EO | surfactant | | |
| Texapon K12 | Sodium Lauryl | Anionic | 7.00 | 7.00 |
| powder | Sulphate C12 | surfactant | | |
| Tinopal CBS-X | | Brightener | 0.50 | 0.50 |
| Savinase 6.0 T, | Protease | Enzyme | 0.40 | 0.40 |
| Type W | | | | |
| Termamyl 120 T | Alpha-Amylase | Enzyme | 0.30 | 0.30 |
| Sodium sulphate | Sodium Sulphate | Filler | 5.50 | 5.50 |
| Perfume oil P1, | | Fragrance | 0.30 | 0.50 |
| P2, P3, P4, P5, | | | | |
| P6, P7, P8, P9, | | | | |
| P10, P11 and P12 | | | | |

### Example F2

### All-Purpose Cleaner

| | | | % by | % by |
|---|---|---|---|---|
| Material | Chemical Name | Function | weight | weight |
| Deionised | Water | Solvent | Ad 100 | Ad 100 |
| water | | | | |
| Mergal K9N | 5-Chloro-2-methyl-3- | Preservative | 0.1 | 0.1 |
| | (2H)-isothiazolone | | | |
| | and 2-methyl-3-(2H)- | | | |
| | isothiazolone | | | |
| Trisodium | Trisodium Citrate | Complex | 3.0 | 3.0 |
| citrate | dihydrate | former | | |
| dihydrate | | | | |
| Zetesol NL-2 | Fatty alcohol C12 14- | Anionic | 30.0 | 30.0 |
| | sulphate, Sodium | surfactant | | |
| Imbentin | Fatty alcohol C12- | Non-ionic | 5.0 | 5.0 |
| C/125/055 | C15, 8EO | surfactant | | |
| Ethanol | Ethanol | Solvent | 2.0 | 2.0 |
| Perfume oil P1, | | Fragrance | 0.3 | 0.5 |
| P2, P3, P4, P5, | | | | |
| P6, P7, P8, P9, | | | | |
| P10, P11 and P12 | | | | |

### Example F3

### Shampoo

| | | % by | % by |
|---|---|---|---|
| Material | INCI-Name | weight | weight |
| Deionised | Water | Ad 100 | Ad 100 |
| water | | | |
| Plantacare PS | Sodium laureth sulphate, | 20.0 | 20.0 |
| 10 | Lauryl glucoside | | |
| Euperlan PK | Glycol distearate, Sodium | 6.0 | 6.0 |
| 771 | lauryl sulphate, Cocamide | | |
| | MEA, Laureth-10 | | |
| Dragocide | Phenoxyethanol, Methylparaben, | 0.5 | 0.5 |
| liquid | Ethylparaben, Butylparaben, | | |
| | Propylparaben, Isobutylparaben | | |
| Sodium chloride | Sodium chloride | 1.4 | 1.4 |
| Citric acid | Citric acid | 0.1 | 0.1 |
| monohydrate | | | |
| crystalline | | | |
| Perfume oil P1, | Fragrance | 0.5 | 0.8 |
| P2, P3, P4, P5, | | | |
| P6, P7, P8, P9, | | | |
| P10, P11 and P12 | | | |

### Example F4

### Shower Gel

| | | % by | % by |
|---|---|---|---|
| Material | INCI-Name | weight | weight |
| Deionised | Water | Ad 100 | Ad 100 |
| water | | | |
| Plantacare PS | Sodium laureth sulphate, Lauryl | 20.0 | 20.0 |
| 10 | glucoside | | |
| Dragocide | Phenoxyethanol, Methylparaben, | 0.5 | 0.5 |
| liquid | Ethylparaben, Butylparaben, | | |
| | Propylparaben, Isobutylparaben | | |
| Sodium chloride | Sodium chloride | 1.4 | 1.4 |
| Citric acid | Citric acid | 1.3 | 1.3 |
| monohydrate | | | |
| crystalline | | | |
| Perfume oil P1, | Fragrance | 0.5 | 0.7 |
| P2, P3, P4, P5, | | | |
| P6, P7, P8, P9, | | | |
| P10, P11 and P12 | | | |

### Example F5

### Fabric Conditioner

| | | | % by | % by |
|---|---|---|---|---|
| Material | Chemical Name | Function | weight | weight |
| Deionised water | Water | Solvent | Ad 100 | Ad 100 |
| Rewoquat WE 18 | Dialkylester- | Cationic | 16.6 | 16.6 |
| | ammonium- | surfactant | | |
| | ethosulphate | | | |
| Mergal K9N | 5-Chloro-2-methyl-3- | Preservative | 0.10 | 0.10 |
| | (2H)-isothiazolone and | | | |
| | 2-methyl-3-(2H)- | | | |
| | isothiazolone | | | |
| Dow Corning | Polydimethyl-siloxane | Defoaming | 0.30 | 0.30 |
| 1520 Antifoam | | agent | | |
| Magnesium | Magnesium chloride | Consistency | 10.00 | 10.00 |
| chloride 1% solution | solution | provider | | |
| Perfume oil P1, | Fragrance | 0.55 | 0.75 | |
| P2, P3, P4, P5, | | | | |
| P6, P7, P8, P9, | | | | |
| P10, P11 and P12 | | | | |

### Example F6

### Eau de Cologne/Eau de Toilette

| | % by | % by |
|---|---|---|
| Ingredients | weight | weight |
| Perfume oil P1, P2, P3, P4, P5, P6, | 5 | 10 |
| P7, P8, P9, P10, P11 and P12 | | |
| Ethanol | Ad 100 | Ad 100 |
| Dye, UV-stabiliser | q.s. | q.s. |
| Water | 18 | 10 |

### Example F7

### Aerosol Pump Spray

| Ingredients | % by weight | % by weight |
|---|---|---|
| Perfume oil P1, P2, P3, P4, P5, P6, | 1.0 | 1.5 |
| P7, P8, P9, P10, P11 and P12 | | |
| Ethanol | Ad 100 | Ad 100 |
| Water | 5.0 | 8.0 |
| Alpha-Tocopherol | 0.20 | 0.20 |
| Hydroxypropyl cellulose | 0.20 | — |
| Rosemary extract, soluble in ethanol | 0.22 | — |
| Cetyl alcohol | 1.00 | 0.50 |

### Example F8

### Shampoo

| | % by | % by | % by |
|---|---|---|---|
| Ingredients | weight | weight | weight |
| Sodium lauryl ether sulphate (e.g. | 12 | 12 | 12 |
| Texapon NSO) | | | |
| Cocamidopropyl betaine (e.g. Dehyton K) | 2 | 2 | 2 |
| Sodium chloride | 1.4 | 1.4 | 1.4 |
| Citric acid | 1.3 | 1.3 | 1.3 |
| Phenoxyethanol, Methyl-, Ethyl-, Butyl- | 0.5 | 0.5 | 0.5 |
| and Propylparaben | | | |
| Perfume oil P1, P2, P3, P4, P5, P6, P7, | 0.3 | 0.5 | 0.7 |
| P8, P9, P10, P11 and P12 | | | |
| Water | Ad 100 | Ad 100 | Ad 100 |

### Example F9

### Washing Powder

| | % by | % by | % by |
|---|---|---|---|
| Ingredients | weight | weight | weight |
| Linear Na-alkylbenzene sulphonate | 8.8 | 8.8 | 8.8 |
| Ethoxylated fatty alcohol C12-18 (7 EO) | 4.7 | 4.7 | 4.7 |
| Na-soap | 3.2 | 3.2 | 3.2 |
| Defoamer DOW CORNING(R) 2-4248S | 3.9 | 3.9 | 3.9 |
| POWDERED ANTIFOAM, silicone oil on | | | |
| Zeolite as the carrier material | | | |
| Zeolite 4A | Ad 100 | Ad 100 | Ad 100 |
| Na-carbonate | 11.6 | 11.6 | 11.6 |
| Na-salt of a copolymer of acrylic acid and | 2.4 | 2.4 | 2.4 |
| maleic acid (Sokalan CP5) | | | |
| Na-silicate | 3.0 | 3.0 | 3.0 |
| Carboxymethylcellulose | 1.2 | 1.2 | 1.2 |
| Dequest | 2.8 | 2.8 | 2.8 |
| 2066([[(Phosphonomethyl)imino]bis[(ethylene- | | | |
| nitrilo)bis(methylene)]]tetrakis-phosphonic acid, | | | |
| sodium salt) | | | |
| Optical brightener | 0.2 | 0.2 | 0.2 |
| Na-sulphate | 6.5 | 6.5 | 6.5 |
| Protease | 0.4 | 0.4 | 0.4 |
| Sodium perborate tetrahydrate | 21.7 | 21.7 | 21.7 |
| Perfume oil P1, P2, P3, P4, P5, P6, | 0.25 | 0.35 | 0.5 |
| P7, P8, P9, P10, P11 and P12 | | | |
| EDTA | 1.0 | 1.0 | 1.0 |

### Example F10

### Liquid Detergent

| | % by |
|---|---|
| Ingredients | weight |
| Deionised water | 39.9 |
| Optical brightener | 0.10 |
| Coconut fatty acids (C12-C18) | 7.5 |
| Potassium hydroxide 50% solution | 4.3 |
| Propane-1,2-diol | 5.00 |
| Fatty alcohols C12-C15, 8 EO | 12.0 |
| Na-salt of secondary alkyl sulphonates (C13-C17) | 17.0 |
| Triethanolamine | 2.0 |
| Trisodium citrate dihydrate | 5.0 |
| Dequest | |
| 2066([[(Phosphonomethyl)imino]bis[(ethylenenitrilo)bis | 3.0 |
| (methylene)]]tetrakis-phosponic acid, Sodium salt) | |
| Ethanol | 3.0 |
| Enzymes | 0.7 |
| Perfume oil P1, P2, P3, P4, P5, P6, P7, P8, P9, P10, | 0.5 |
| P11 and P12 | |

### Example F11

### Liquid Detergent Concentrate

| | % by |
|---|---|
| Ingredients | weight |
| Deionised water | 13.4 |
| Coconut fatty acids (C12-C18) | 10.0 |
| Fatty alcohols C12-C15, 8 EO | 26.0 |
| Na-salt of secondary alkyl sulphonates (C13-C17) | 26.5 |
| Triethanolamine | 8.5 |
| Na-salt of fatty alcohol sulphates C12-C14 | 3.0 |
| Ethanol | 5.5 |
| Urea | 4.5 |
| Enzymes | 0.9 |
| Citric acid | 1.0 |
| Perfume oil P1, P2, P3, P4, P5, P6, P7, P8, P9, | 0.7 |
| P10, P11 and P12 | |

### Example F12

### Deodorant Formulation in the Form of a Roll-On Gel

| | % by | % by |
|---|---|---|
| Component/NAME | weight | weight |
| 1,3-Butylene glycol | 2.00 | 2.00 |
| PEG-40-hydrogenated castor oil | 2.00 | 2.00 |
| Hydroxyethyl cellulose | 0.50 | 0.50 |
| Perfume oil P1, P2, P3, P4, P5, P6, | 0.60 | 0.75 |
| P7, P8, P9, P10, P11 and P12 | | |
| Phenoxyethanol (preservative) | 0.30 | — |
| Water | ad 100.00 | ad 100.00 |

### Example F13

### Cream

| | % by | % by |
|---|---|---|
| Component/NAME | weight | weight |
| Paraffin oil | 10.00 | 10.00 |
| Ozokerite | 4.00 | 4.00 |
| Vaseline | 4.00 | 4.00 |
| Vegetable oil | 10.00 | 10.00 |
| Wool wax alcohol | 2.00 | 2.00 |
| Aluminium stearate | 0.40 | 0.40 |
| Perfume oil P1, P2, P3, P4, P5, P6, | 0.80 | 1.00 |
| P7, P8, P9, P10, P11 and P12 | | |
| 1,2-Pentanediol | 2.00 | 2.00 |
| Water | ad 100.00 | ad 100.00 |

### Example F14

### Oil-in-Water Lotion

| | % by | % by |
|---|---|---|
| Component/NAME | weight | weight |
| Paraffin oil | 5.00 | 5.00 |
| Isopropyl palmitate | 5.00 | 5.00 |
| Cetyl alcohol | 2.00 | 2.00 |
| Beeswax | 2.00 | 2.00 |
| Ceteareth-20 | 2.00 | 2.00 |
| PEG-20-Glyceryl stearate | 1.50 | 1.50 |
| Glycerol | 3.00 | 3.00 |
| Perfume oil P1, P2, P3, P4, P5, P6, | — | 0.80 |
| P7, P8, P9, P10, P11 and P12 | | |
| Parabens | 0.30 | 0.30 |
| Water | ad 100.00 | ad 100.00 |

### Example F15

### Air Freshener in Gel Form

| | % by | % by |
|---|---|---|
| Component/NAME | weight | weight |
| Demineralised Water | Ad 100.00 | Ad 100.00 |
| Genugel ® X-6424 (1) (Carrageenan) | 2.00 | 2.00 |
| Perfume oil P1, P2, P3, P4, P5, P6, P7, P8, P9, P10, | 0.80 | 1.20 |
| P11 and P12 | | |
| Arkopal ® N 100 or Tergitol ® NP 10 (emulsifier) | 3.50 | 3.50 |
| Preventol ® D (preservative) | 5.00 | 5.00 |

### Example F16

### Air Freshener in Gel Form

5 g Accurel (porous homo-polypropylene powder with a 75% cavity fraction, product of Akzo Nobel Faser AG, Obernburg, Germany) are loaded with 15 g of the perfume oil, perfume oil P1, P2, P3, P4, P5, P6, P7, P8, P9, P10, P11 and P12 by mixing the constituents under a vacuum. The resulting powder is then stirred at normal pressure with 4.5 g water, so mixture M1 is obtained. In a separate vessel, 2.5 g carrageenan, 0.3 g chloroacetamide and 0.5 g calcium chloride dihydrate are dissolved in 62 g water while heating at a maximum of 75° C. Mixture M1 is introduced into this solution while stirring and homogenised. The resulting, preferably still warm mixture is poured into the desired mould (spheres, semi-spheres, pads, cylinders, cuboids, cubes, shells or the like). After cooling to about 20° C., air fresheners in gel form are obtained, the loading of which with the respective perfume oil being about 20% by weight based on the total weight of the air freshener.

### Example F17

### Setting Lotion

| | % by | % by |
|---|---|---|
| Component/NAME | weight | weight |
| Polyvinylpyrrolidone/Vinyl acetate/ | 5.00 | 5.00 |
| Vinyl propionate copolymer | | |
| Ethanol | 45.00 | 45.00 |
| Perfume oil P1, P2, P3, P4, P5, | 0.50 | 0.60 |
| P6, P7, P8, P9, P10, P11 and P12 | | |
| Preservatives | 1.50 | 1.50 |
| Water | ad 100.00 | ad 100.00 |

### Example F18

### Care Mask

| | % by | % by |
|---|---|---|
| Component/NAME | weight | weight |
| PEG-50 Lanolin | 0.50 | 0.50 |
| Glyceryl stearate | 2.00 | 2.00 |
| Sunflower seed oil | 3.00 | 3.00 |
| Bentonite | 8.00 | 8.00 |
| Kaolin | 35.00 | 35.00 |
| Zinc oxide | 5.00 | 4.00 |
| Perfume oil P1, P2, P3, P4, P5, | 0.35 | 0.50 |
| P6, P7, P8, P9, P10, P11 and P12 | | |
| Preservatives | q.s. | q.s. |
| Water | ad 100.00 | ad 100.00 |

### Example F19

### Skin Oil

| | % by | % by |
|---|---|---|
| Component/NAME | weight | weight |
| Cetyl palmitate | 3.00 | 3.00 |
| C₁₂₋₁₅−Alkyl benzoate | 2.00 | 2.00 |
| Polyisobutene | 10.00 | 10.00 |
| Squalane | 2.00 | 2.00 |
| Perfume oil P1, P2, P3, P4, P5, | 0.50 | 0.40 |
| P6, P7, P8, P9, P10, P11 and P12 | | |
| Preservatives | q.s. | q.s. |
| Paraffin oil | ad 100.00 | ad 100.00 |

### Example F20

### Hair Conditioner with UV-Protection

| | | % by | % by |
|---|---|---|---|
| Component | INCI Name | weight | weight |
| Renex PEG 6000 | PEG-150 | 2.50 | 2.50 |
| Hair Conditioner Base | Cetyl alcohol, Behentrimonium | 3.00 | 3.00 |
| | chloride, Triticum vulgare (Wheat) | | |
| | Bran extract, Linoleic acid | | |
| PCL-Solid | Stearyl heptanoate, Stearyl | 0.50 | 0.50 |
| | caprylate | | |
| Dow Corning 5200 | Laurylmethicone copolyol | 0.50 | 0.50 |
| Natrosol 250 HR | Hydroxyethylcellulose | 0.50 | 0.50 |
| Benzophenone-4 | Benzophenone-4 | 1.00 | 0.50 |
| Neo Heliopan AP | Disodium phenyl dibenzimidazole | 1.00 | 0.80 |
| | tetrasulfphonate | | |
| Amino methyl propanol | Amino methyl propanol | 2.00 | 1.20 |
| Nipagin M | Methylparaben | 0.30 | 0.30 |
| Dow Corning 949 Cationic | Amodimethicone, Cetrimonium | 2.00 | 2.00 |
| emulsion | chloride, Trideceth-12 | | |
| Perfume oil P1, P2, P3, P4, | Perfume | 0.80 | 1.00 |
| P5, P6, P7, P8, P9, P10, | | | |
| P11 and P12 | | | |
| Water | Water (Aqua) | Ad 100 | Ad 100 |

### Example F21

### Deodorant Sticks

| Component/NAME | % by weight | % by weight |
|---|---|---|
| Sodium stearate | 8.00 | 8.00 |
| PPG-3 Myristyl ether | 70.00 | 70.00 |
| 1,2-Propylene glycol | 10.00 | 10.00 |
| 1,1-Dimethyl-3-phenylpropanol | 0.20 | — |
| 2-Butyloctanoic acid | — | 0.20 |
| Perfume oil P1, P2, P3, P4, P5, P6, | 0.55 | 0.65 |
| P7, P8, P9, P10, P11 and P12 | | |
| Water | Ad 100 | Ad 100 |

### Example F22

### Micro Emulsion Gels

| | % by | % by |
|---|---|---|
| Component/NAME | weight | weight |
| Glyceryl isostearate | 1.80 | 2.00 |
| Octoxy glycerine | 1.00 | 0.80 |
| Ceteareth-15 | 5.20 | 5.00 |
| PEG-150 Distearate | 1.00 | 1.00 |
| Aluminium chlorohydrate | 5.00 | 5.00 |
| Isotridecylisononanoate | 3.30 | 3.50 |
| Cyclomethicone | 6.60 | 6.40 |
| Perfume oil P1, P2, P3, P4, P5, P6, | 0.60 | 0.70 |
| P7, P8, P9, P10, P11 and P12 | | |
| Water | Ad 100 | Ad 100 |

### Example F23

### Antiperspirant Formulations

| | % by | % by |
|---|---|---|
| Component/NAME | weight | weight |
| Reach AZP-908 SUF (aluminium-zirconium tetrachlorohydrexgly) | 24.00 | 22.00 |
| Cyclomethicone (Pentamer) | Ad 100 | Ad 100 |
| Polydecene (Silkflo 364 NF) | 17.50 | 20.00 |
| Neo Heliopan OS (Ethylhexyl salicylate, Symrise) | 2.50 | 1.00 |
| L-Menthyl lactate (Frescolat ML, Symrise) | 0.25 | — |
| Polyethylene | 3.00 | 3.00 |
| Hydrogenated castor oil | 2.00 | 2.00 |
| Promyristyl PM-3 | 7.00 | 7.00 |
| PEG-8 Distearate | 3.00 | 3.00 |
| Silicon dioxide (Cab-O-Sil M-5) | 1.00 | 1.00 |
| Stearyl alcohol | 15.00 | 10.00 |
| Octyldodecanol | — | 8.00 |
| Perfume oil P1, P2, P3, P4, P5, P6, P7, P8, P9, P10, P11 and P12 | 0.75 | 0.95 |

### Example F24

### Suspension Sticks

| | % by | % by | % by |
|---|---|---|---|
| Component/NAME | weight | weight | weight |
| Stearyl alcohol | 20.00 | 20.00 | 20.00 |
| Cyclomethicone | Ad 100 | Ad 100 | Ad 100 |
| PPG-14 Butyl ether | 2.00 | 2.00 | 2.00 |
| Hydrogenated castor oil | 1.00 | 1.00 | 1.00 |
| Talc | 2.00 | 2.00 | 2.00 |
| Aluminium chlorohydrate, powder | 20.00 | 20.00 | 20.00 |
| Triclosan ® (5-Chloro-2-(2,4- | 0.30 | — | 0.30 |
| dichlorophenoxy)phenol) | | | |
| Ethylhexylglycerin (Octoxyglycerin) | 0.50 | 0.80 | 0.50 |
| 1,1-Dimethyl-3-phenylpropanol | 0.30 | 0.25 | 0.35 |
| Anisic alcohol | — | — | 0.15 |
| Perfume oil P1, P2, P3, P4, P5, P6, | 0.90 | 0.85 | 0.75 |
| P7, P8, P9, P10, P11 and P12 | | | |

### Example F25

### Deodorant Spray

| | % by | % by |
|---|---|---|
| Component/NAME | weight | weight |
| PEG-40 Hydrogenated castor oil | 3.00 | 3.00 |
| Ethylhexylglycerin (Octoxyglycerin) | 0.80 | 0.80 |
| Ethanol | 40.00 | 40.00 |
| Citrate buffer | 0.50 | 0.50 |
| 1,2-Hexanediol/1,2-Octanediol (1:1) | 0.50 | 0.25 |
| Phenoxyethanol | | 0.35 |
| Triclosan ® (5-Chlor-2-(2,4- | 0.25 | — |
| dichlorphenoxy)phenol) | | |
| 2-Benzylheptan-1-ol (Jasmol) | — | 0.05 |
| Perfume oil P1, P2, P3, P4, P5, P6, | 0.90 | 0.75 |
| P7, P8, P9, P10, P11 and P12 | | |
| Water | Ad 100 | Ad 100 |

1. A fragrance substance mixture comprising
(a) compound (A-1) and compound (A-2) 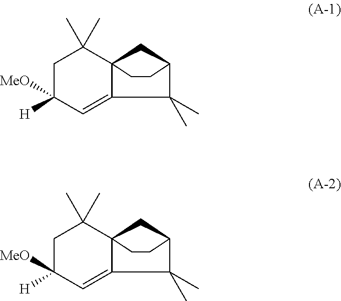 [SMILES: CO[C@H]1C=C2C(C)(C)[C@H]3CC[C@@]2(C3)C(C)(C)C1 ; CO[C@@H]1C=C2C(C)(C)[C@H]3CC[C@@]2(C3)C(C)(C)C1]
and
(b) one or more further musk fragrance substances, which are not isolongifolenyl methyl ethers,
wherein the mass ratio of the total quantity of musk fragrance substances of constituent (b) to the total quantity of compounds (A-1) and (A-2) (constituent (a)) is greater than or equal to 1:2.

2. The fragrance substance mixture according to claim 1, wherein the mass ratio of the total quantity of musk fragrance substances of constituent (b) to the total quantity of compounds of constituent (a) is in the range from 3:2 to 20:1.

3. The fragrance substance mixture according to claim 1, wherein the further musk fragrance substance(s) of constituent (b) are selected from the group consisting of macrocyclic musk fragrance substances, nitro-musk fragrance substances, polycyclic musk fragrance substances and alicyclic musk fragrance substances.

4. The fragrance substance mixture according to claim 1, wherein the mass ratio of the total quantity of the isomer (A-1) to the total quantity of the isomer (A-2) is greater than or equal to 11:4.

5. The fragrance substance mixture according to claim 1, further comprising
(c) or more terpene alcohols having the summation formula C₁₀HₓO, wherein x signifies the number 18, 20 or 22
and/or
(d) or more further woody fragrance substances, wherein the further woody fragrance substance or the further woody fragrance substances of constituent (d) is or are different from constituent (a) and constituent (b).

6. The fragrance substance mixture according to claim 5, wherein the mass ratio of the total quantity of terpene alcohols (constituent (c)) to the total quantity of compounds (A-1) and (A-2) (constituent (a)) is greater than or equal to 1:1.

7. The fragrance substance mixture according to claim 5, wherein constituent (d) comprises one more further woody fragrance substances selected from
(d)(i) 2,3,8,8-Tetramethyl-1,2,3,4,5,6,7,8-octahydro-2-naphthalenyl methyl ketone and/or 1-[1,2,3,4,6,7,8,8a-octahydro-1,2,8,8-tetramethylnaphtalen-2-yl]ethanone
and/or
(d)(ii) sandalwood fragrance substances.

8. The fragrance substance mixture according to claim 7, wherein the mass ratio of the total quantity of constituent (d)(i) 2,3,8,8-Tetramethyl-1,2,3,4,5,6,7,8-octahydro-2-naphthalenyl methyl ketone and 1-[1,2,3,4,6,7,8,8a-octahydro-1,2,8,8-tetramethylnaphtalen-2-yl]ethanone to the total quantity of compounds (A-1) and (A-2) (constituent (a)) is greater than or equal to 1:1.

9. The fragrance substance mixture according to claim 7, wherein the mass ratio of the total quantity of sandalwood fragrance substances (constituent (d)(ii)) to the total quantity of compounds (A-1) and (A-2) (constituent (a)) is greater than or equal to 1:1.

10. The fragrance substance mixture according to claim 1, wherein constituent
(b) comprises one or more macrocyclic musk fragrance substances.

11. The fragrance substance mixture according to claim 1, wherein constituent
(b) comprises 15-pentadec-(11/12)-enolide, ethylene brassylate, 15-pentadecanolide, 5-cyclohexadecen-1-one, 7-cyclohexadecen-1-one, 8-cyclohexadecen-1-one, 7/8-cyclohexadecen-1-one, cyclohexadecanone, 3-methylcyclopentadecanone, 3-methylcyclopentadecenone and/or 9-cycloheptadecen-1-one.

12. A perfumed product containing the fragrance substance mixture, according to claim 1.

13. The perfumed product according to claim 12, wherein the perfumed product is selected from the group consisting of washing and cleaning agents and hygiene and care products.

14. The fragrance substance mixture according to claim 1, wherein the total mass of the compounds (A-1) and (A-2) (constituent (a)) is in the range from 0.25 to 10% by weight in each case based on the total mass of all the fragrance substances contained in the fragrance substance mixture.

15. A method for modifying a metallic note of one or more musk compounds comprising the following step:
mixing one or more musk compounds with a total quantity of compounds (A-1) and (A-2) (constituent (a)), as defined in claim 1, which is sufficient to modify the metallic note of one, several or all the musk compounds.

16. The fragrance substance mixture according to claim 1, wherein the mass ratio of the total quantity of the isomer (A-1) to the total quantity of the isomer (A-2) is in the range from 3:1 to 12:1.

17. The fragrance substance mixture according to claim 5, wherein one or more of the terpene alcohols are selected from the group consisting of linalool, eucalyptol, dihydromyrcenol, citronellol, nerol, geraniol, menthol, tetrahydro linalool and tetrahydro geraniol.

18. The fragrance substance mixture according to claim 7, wherein the (d)(ii) sandalwood fragrance substances are selected from the group consisting of 2-methyl-4-(2,2,3-trimethyl-3-cyclopenten-1-yl)butanol, 5-(2,2,3-trimethyl-3-cyclopenten-1-yl)-3-methylpentan-2-ol, 2-ethyl-4-(2,2,3-trimethyl-3-cyclopenten-1-yl)-2-buten-1-ol, 3-methyl-5-(2,2,3-trimethyl-3-cyclopenten-1-yl)-4-penten-2-ol, 3,3-dimethyl-5-(2,2,3-trimethyl-3-cyclopenten-1-yl)-4-penten-2-ol, 3-isocamphylcyclohexanol and [1-methyl-2-(1,2,2-trimethyl[3.1.0]hex-3-ylmethyl)cyclopropyl]methanol.

19. The perfumed product according to claim 12, wherein the fragrance substance mixture is present in a sensorily effective quantity.

20. The perfumed product according to claim 12, wherein the total mass of the compounds (A-1) and (A-2) (constituent (a)) is in the range from 0.25 to 10% by weight, in each case based on the total mass of all the fragrance substances contained in the perfumed product.
